(19) United States
(12) Patent Application Publication
(10) Pub. No.: US 2025/0144250 A1
(43) Pub. Date: May 8, 2025

(54) PH-SENSITIVE FLUOROPHORES

(71) Applicants: The General Hospital Corporation, Boston, MA (US); Georgia State University Research Foundation, Inc., Atlanta, GA (US)

(72) Inventors: Hak Soo Choi, Needham, MA (US); Satoshi Kashiwagi, Boston, MA (US); Maged Henary, Atlanta, GA (US)

(21) Appl. No.: 18/730,243

(22) PCT Filed: Jan. 31, 2023
(86) PCT No.: PCT/US2023/012049

§ 371 Date: Jul. 18, 2024

Related U.S. Application Data
(60) Provisional application No. 63/304,918, filed on Jan. 31, 2022.

(51) Int. Cl. A61K 49/00; C09B 23/01

(52) U.S. Cl. CPC A61K 49/0032; A61K 49/006; C09B 23/0066

(57) ABSTRACT

The present application provides pH-sensitive fluorophores, pharmaceutical compositions comprising pH-sensitive fluorophores, and methods for imaging of cancerous tumors comprising administering to the subject an effective amount of a compound or a pharmaceutical composition, waiting a time sufficient to allow the compound to accumulate in the cancerous tumor to be imaged, and imaging the cancerous tumor with a fluorescence imaging technique. Further provided methods of treating cancer, comprising imaging a cancerous tumor in a subject; and surgically removing the cancerous tumor from the subject.

### CLAIM OF PRIORITY

This application claims priority to U.S. Patent Application Ser. No. 63/304,918, filed on Jan. 31, 2022, the entire contents of which are hereby incorporated by reference.

### FEDERALLY SPONSORED RESEARCH OR DEVELOPMENT

This invention was made with Government support under Grant Nos. EB022230 and HL 143020 awarded by the National Institutes of Health. The Government has certain rights in the invention.

### TECHNICAL FIELD

This invention relates to heptamethine cyanine-based fluorophores useful, e.g., in intraoperative optical imaging and image-guided cancer surgeries.

### BACKGROUND

There are numerous deadly diseases affecting current human population. For example, cancer is one of the leading causes of death in contemporary society. Currently, cancer incidence is nearly 450 cases of cancer per 100,000 men and women per year, while cancer mortality is nearly 71 cancer deaths per 100,000 men and women per year. The socioeconomic burden of cancer is substantial and reflects both healthcare spending as well as lost productivity due to co-morbidities and premature death. Healthcare spending on treating cancer exceed tens of billions of dollars worldwide. However, the economic burden of lost productivity due to cancer is over 60% of the total economic burden associated with cancer. Prevention, early detection, and effective treatment help reduce this economic burden.

### SUMMARY

Fluorescence-guided surgery (“FGS”) aids surgeons with real-time visualization of small cancer foci and borders, which improves surgical and prognostic efficacy of cancer. Despite the steady advances in imaging devices, there is a scarcity of fluorophores available to achieve optimal FGS. This disclosure is based, at least in part, on a realization that NIR fluorophores containing an amino group near conjugated double bonds of a cyanine dye are “activatable” by detecting mildly low pH at a tumor site based on photoinduced electron transfer (PeT). Hence, the present disclosure provides pH-sensitive near-infrared fluorophores that exhibit rapid signal changes in acidic tumor microenvironments (TME) and mitochondrial and lysosomal retention. As the experimental data provided in this disclosure demonstrates, after topical application on peritoneal tumor regions in ovarian-cancer bearing mice, a rapid fluorescence increase (less than about 10 min) and extended preservation of signals (over about 4 hours post-topical application) were observed, which together allow for visualization of submillimeter tumors with high tumor-to-background ratio (greater than about 5). The compounds within the present claims are permeable to cancer cells, e.g., via organic anion transporter peptides and co-localize in the mitochondria and lysosomes due to the positive charges, advantageously allowing for a long retention time during FGS. Hence, the fluorophore compounds within the present claims have a significant impact on surgical and diagnostic applications for the treatment of malignant tumors. An example of an application of a fluorophore compound of this disclosure to selectively image ovarian cancer is shown in FIG. 1E. Referring to FIG. 1E, a compound containing an ionizable amino group is taken up by tumor cells via organic anion transporters (OATPs), which are often overexpressed in cancer cells. Photoinduced electron transfer (PeT) occurs in the acidic tumor microenvironment (TME) and lysosomes of cancer cells, where the amino group of the fluorophore compound is protonated and fluorescence emission is enhanced. These features are optimal for rapid fluorescence recovery, durable retention, and excellent targetability upon topical application, allowing for intraoperative imaging for a sufficiently long time and the detection of, e.g., small peritoneal dissemination of ovarian cancer.

In some embodiments, the present disclosure provides a compound of Formula (I):

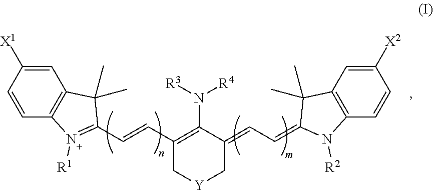

or a pharmaceutically acceptable salt thereof, wherein:
Y is selected from CH₂, O, and S;
X¹ and X² are each independently selected from H, halogen, CN, NO₂, OH, NH₂, C₁₋₃ alkyl, C₁₋₃ haloalkyl, C₁₋₃ alkoxy, C₁₋₃ haloalkoxy, C₁₋₃ alkylamino, and di(C₁₋₃ alkyl)amino;
n is 1 or 2; m is 1 or 2;
R¹ and R² are each independently selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl;
R³ and R⁴ are each independently selected from H, C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl;
provided that at least one of R³ and R⁴ is other than H; or
R³ and R⁴ together with the N atom to which they are attached form a ring of formula:

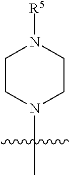

wherein R⁵ is selected from H, C₁₋₈ alkyl, C₁₋₈ haloalkyl, C(═O)Cy¹, and S(═O)₂Cy¹, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, or Cy¹; and
each Cy¹ is independently selected from C₆₋₁₀ aryl and 5-10 membered heteroaryl, each of which is optionally substituted with 1 or 2 substituents independently selected from NO₂, CN, OH, C₁₋₃ alkoxy, C(═O)OH, NH₂, C₁₋₃ alkylamino, and di(C₁₋₃ alkyl)amino.

In some embodiments, the compound has formula:

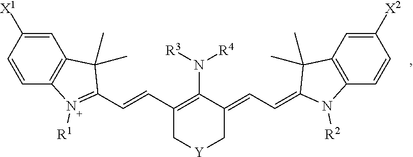

or a pharmaceutically acceptable salt thereof.

In some embodiments, n is 1 and m is 2, or n is 2 and m is 1.

In some embodiments, Y is CH₂.

In some embodiments, Y is O.

In some embodiments, Y is S.

In some embodiments, R¹ is C₁₋₈ alkyl, optionally substituted with OH, C(═O)OH, SO₃H, or C₆₋₁₀ aryl.

In some embodiments, R² is C₁₋₈ alkyl, optionally substituted with OH, C(═O)OH, SO₃H, or C₆₋₁₀ aryl.

In some embodiments, X¹ is H and X² is H.

In some embodiments, X¹ is halogen and X² is halogen.

In some embodiments, R³ or R⁴ is C₁₋₈ alkyl, optionally substituted with OH, C(═O)OH, SO₃H, or C₆₋₁₀ aryl.

In some embodiments, R³ and R⁴ together with the N atom to which they are attached form a ring of formula:

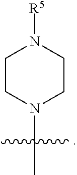

In some embodiments, R⁵ is selected from H and S(═O)₂Cy¹.

In some embodiments, the compound is selected from any one of the following compounds:

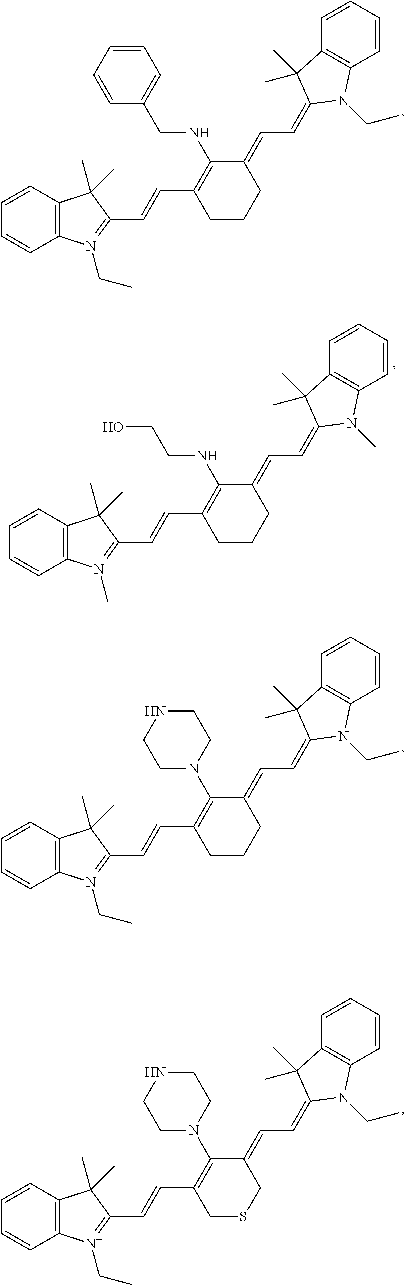 [SMILES: CCN1/C(=C/C=C2\CCCC(/C=C/C3=[N+](CC)c4ccccc4C3(C)C)=C2NCc2ccccc2)C(C)(C)c2ccccc21 ; CN1/C(=C/C=C2\CCCC(/C=C/C3=[N+](C)c4ccccc4C3(C)C)=C2NCCO)C(C)(C)c2ccccc21 ; CCN1/C(=C/C=C2\CCCC(/C=C/C3=[N+](CC)c4ccccc4C3(C)C)=C2N2CCNCC2)C(C)(C)c2ccccc21 ; CCN1/C(=C/C=C2\CSCC(/C=C/C3=[N+](CC)c4ccccc4C3(C)C)=C2N2CCNCC2)C(C)(C)c2ccccc21]

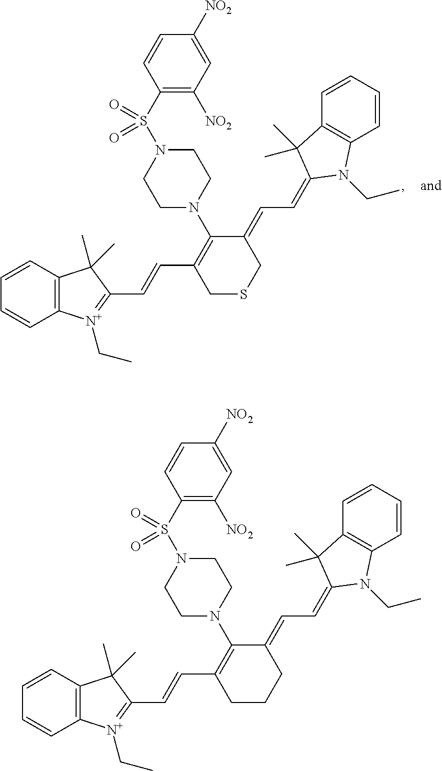 [SMILES: CCN1/C(=C/C=C2\CSCC(/C=C/C3=[N+](CC)c4ccccc4C3(C)C)=C2N2CCN(S(=O)(=O)c3ccc([N+](=O)[O-])cc3[N+](=O)[O-])CC2)C(C)(C)c2ccccc21 ; CCN1/C(=C/C=C2\CCCC(/C=C/C3=[N+](CC)c4ccccc4C3(C)C)=C2N2CCN(S(=O)(=O)c3ccc([N+](=O)[O-])cc3[N+](=O)[O-])CC2)C(C)(C)c2ccccc21]

or a pharmaceutically acceptable salt thereof.

In some embodiments, the present disclosure provides a pharmaceutical composition comprising a compound of Formula (I), or a pharmaceutically acceptable salt thereof, and a pharmaceutically acceptable carrier.

In some embodiments, the present disclosure provides a method of imaging a cancerous tumor in a subject, the method comprising:
i) administering to the subject an effective amount of a compound of any one of Formula (I), or a pharmaceutically acceptable salt thereof, or a pharmaceutical composition comprising same;
ii) waiting a time sufficient to allow the compound to accumulate in the cancerous tumor to be imaged; and
iii) imaging the cancerous tumor with a fluorescence imaging technique.

In some embodiments, said administering comprises topically spraying the cancerous tumor or a site of an organ or tissue comprising the cancerous tumor.

In some embodiments, the fluorescence imaging technique is NIR-II fluorescence imaging.

In some embodiments, the time sufficient to allow the compound to accumulate in the cancerous tumor is from about 1 minute to about 24 hours.

In some embodiments, the present disclosure provides a method of treating cancer, the method comprising:
i) imaging a cancerous tumor in a subject according to the method of imaging a cancerous tumor in a subject as described herein; and
ii) surgically removing the cancerous tumor from the subject.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIGS. 1A-1D Chemical structures, physicochemical properties, and optical properties of pH sensing NIR fluorophores: (a) Chemical structures of pH sensing and non-sensing NIR fluorophores. (b) Optical properties of representative fluorophores. pH-dependent changes in absorption and fluorescence emission were determined in phosphate-buffered saline (PBS) with 5% bovine serum albumin (BSA). (c) Proposed pH-dependent intramolecular photoinduced electron transfer (PeT) of the candidate PH probes. (d) Schematic illustration of pH-dependent intramolecular PeT and HOMO and LUMO energy levels of PH03 and PH08 based on density functional theory calculations. The HOMO and LUMO energy levels were plotted based on the optimized SO and S1 geometries using Gaussian 16 at B3-LYP/6-31G (d).

FIG. 1E schematically shows that the compounds of this disclosure are taken up by tumor cells via organic anion transporters (OATPs), which are often overexpressed in cancer cells. Photoinduced electron transfer (PeT) occurs in the acidic tumor microenvironment (TME) and lysosomes of cancer cells, where the heterocyclic amine of the compound is protonated and fluorescence emission is enhanced.

FIG. 2 Cellular uptake of pH sensing NIR fluorophores via membrane transporters: In vitro tumor cell uptake of the pH sensing NIR probes under normoxic and hypoxic conditions. Murine cancer cell line ID8 cells were incubated at normoxic or hypoxic conditions (1% O₂) for 24 h at 37° C. and then incubated for 30 min in the presence of 0.1 μM NIR probes. (a) Representative fluorescence images of cells are shown. The contrast was normalized across all images. Scale bar=100 μm. (b) Quantitative time-course measurements of the fluorescence intensity in cells. *P<0.05, **P<0.01 by two-way ANOVA followed by Tukey's multiple comparisons test (Normoxia vs. Hypoxia). Error bars show mean #s.d. (c) Inhibition assay of transporters to determine the entry mechanisms in ID8 cells. ID8 cells were cultured and incubated with a transporter inhibitor. Cells were pre-blocked with bromsulphthalein (BSP, OATP inhibitor), D22 (OCT inhibitor), or both inhibitors for 10 min and then incubated with 0.2 μM PH08 in 10% FBS for 15 min. Control cells were incubated at 4° C. for 30 min. Cells were then imaged using an epifluorescence microscope. Representative fluorescence images of cells are shown. The contrast was normalized across all images. Scale bar=100 μm. (d) Quantitative measurements of the fluorescence intensity in cells. *P<0.05 by two-way ANOVA followed by Tukey's multiple comparisons test. Error bars show means±s.d.

FIG. 3 Subcellular localization of pH sensing NIR fluorophores: (a) Subcellular localization of each probe (10 μM) in ID8 cells was determined after 30 min of incubation at 37° C. Nuclei, mitochondria, and lysosomes were stained with NucBlue, MitoTracker Green, or LysoTracker Red, respectively. Representative fluorescence images of cells. Scale bar=20 μm. (b) Co-localization index of each probe with MitoTracker Green or LysoTracker Red. The index was calculated by dividing the area of overlap between pH probe and MitoTracker or LysoTracker by the total area of MitoTracker or LysoTracker, respectively. The co-localization index was determined in 5-6 photographic areas (70×92 μm² each) for each probe. *P<0.05 by two-way ANOVA followed by Tukey's multiple comparisons test. Error bars show means±s.d.

FIG. 4 In vivo evaluation of tumor cell targetability of pH probes in a mouse model of ovarian cancer: (a) Intraoperative color and NIR fluorescence images of the abdominal cavity and peritoneal dissemination of ovarian cancer and major organs 4 h post-intraperitoneal injection of 20 nmol of PH03 or PH08. Scale bar=5 mm. (b) Quantitative analysis of target-to-background ratio (TBR) of peritoneal tumors and major organs for each NIR fluorophore. TBR was determined by comparing the signals of tumors (Tu) or organs against muscle (Mu). Du, duodenum; He, heart; In, intestine; Ki, kidneys; Li, liver; Lu, lungs; Pa, pancreas; Sp, spleen; St, stomach (n=3, mean±s.e.m.). (c) Quantitative time-course assessment of TBR for up to 240 min post-injection (n=3, mean±s.e.m.). (d) Histological analysis of tumor targetability of PH08 in orthotopic ovarian cancer model. Representative hematoxylin and eosin (H&E) and corresponding NIR fluorescence images are shown. Mu, muscle; Tu, tumor. Left and right panels: scale bars=500 μm and 50 μm, respectively.

FIG. 5A shows optical properties of candidate fluorophores. pH dependent changes in absorption and fluorescence emission were determined in phosphate-buffered saline (PBS) with 5% bovine serum albumin (BSA).

FIG. 5B-5C b) Quenching patterns of PH08 and ICG in terms of concentration measured using the FLARE imaging system after incubating the fluorophore in 5% BSA. (c) Photobleaching curves were obtained by incubating different concentrations of PH08 in 5% BSA for 3 h under continuous laser irradiation.

FIG. 6 In vivo evaluation of tumor cell targetability of pH probes in a mouse model of ovarian cancer. (a) Intraoperative color and NIR fluorescence images of peritoneal tumors 6 h post-intraperitoneal injection of 20 nmol of PH08. Scale bar=5 mm. (b) Quantitative analysis of target-to-background ratio (TBR) at 60 min post-injection (n=3, mean±s.e.m.).

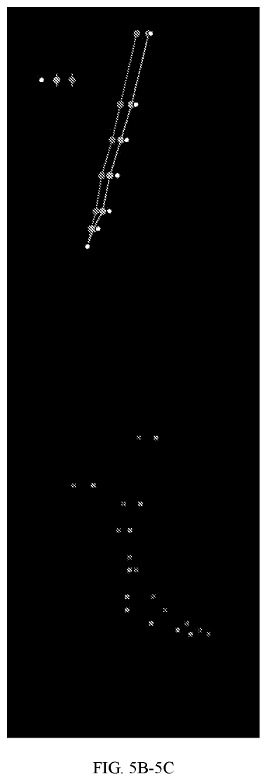

FIG. 7 Cellular uptake of pH sensing NIR fluorophores via membrane transporters and retention in organelles. (a) Inhibition assay of cellular uptake of PH08 to determine the entry mechanisms in ID8 cells. Cultured cells were incubated with bromsulphthalein (BSP), cyclosporin A, MK-571, D22, 2DG, oligomycin, Dyngo 4a, Pitstop 2 for 5-30 min, and then incubated with 0.2 μM PH08 in 10% or 0% FBS media for 15 min. Alternatively, cells were incubated at 4° C. for 30 min. Cells were then imaged using an epifluorescence microscope. Quantitative measurements of the fluorescence intensity in cells. *P<0.05, ****P<0.0001 by one-way ANOVA followed by Tukey's multiple comparisons test. Error bars show means±s.d. (b) Subcellular localization of each probe (10 μM) in ID8 cells was determined after 30 min of incubation at 37° C. Nuclei, mitochondria, and lysosomes were stained with NucBlue, MitoTracker Green, or LysoTracker Red, respectively. Representative fluorescence images of cells. Scale bar=20 μm.

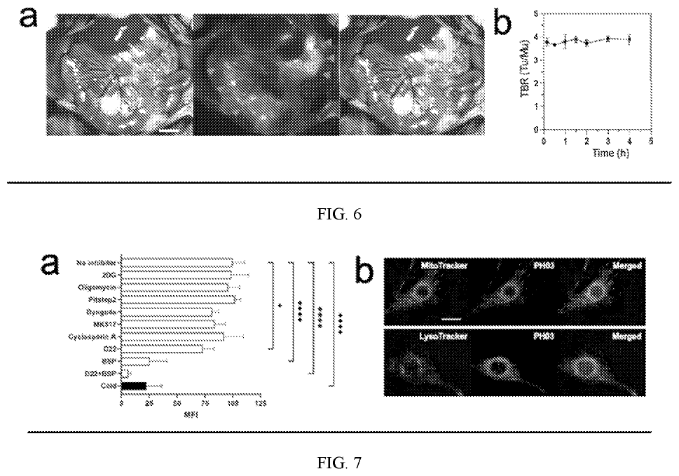

FIG. 8 Ratiometric fluorescence imaging by chemical modification of cyclohexene: PH09 shows enhanced optophysical properties of pH sensing. Chemical structures of pH sensing fluorophore PH09, and its absorbance, fluorescence spectra were measured in PBS with 5% BSA.

FIG. 9 shows additional details of optical properties of candidate fluorophores PH03, PH08, PH09, and PH10.

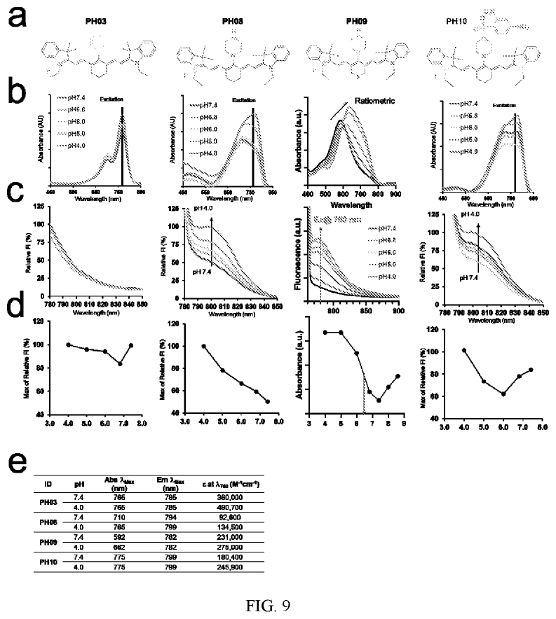

FIG. 10 Tumor cell targetability and cytotoxicity of the pH sensing NIR fluorophore. (a) Cytotoxicity of the fluorophore. Cells were treated with 0-25 UM of PH08 for 24 h, followed by an assessment of cell viability using the Cell Counting Kit-8 (CCK-8) (n=3, mean±s.d.). (b) Murine and human ovarian cancer cell lines including ID8 cells and SKOV3 cells, NIH/3T3 fibroblast, and C2Cl2 muscle cells were cultured and incubated at 37° C. for 15 min in the presence of 0.2 μM PH08 and imaged under the epifluorescence NIR microscope. Quantitative measurements of the fluorescence intensity of cells in 10% FBS (n=3, mean #s.d.). Scale bar=100 μm. ns, not significant, *P<0.05, **P<0.01, ***P<0.001, ****P<0.0001 by one-way ANOVA followed by Tukey's multiple comparisons test.

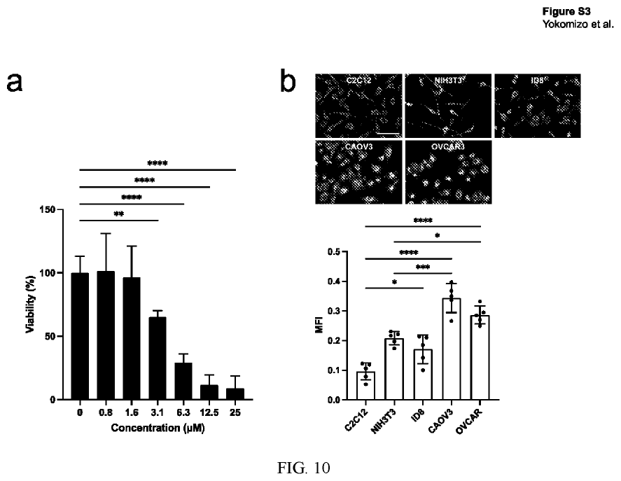

FIG. 11 Cellular uptake of pH sensing NIR fluorophores via membrane transporters in a human ovarian cancer cell line. In vitro tumor cell uptake of the pH sensing NIR probes with or without serum in media under normoxic and hypoxic conditions. An inhibition assay of cellular uptake of PH08 was performed to determine the entry mechanisms. SKOV-3 human cancer cells were incubated under normoxic and hypoxic (1% O2) conditions. Cultured cells were incubated with BSP, D22, or both inhibitors for 10 min and then incubated with 0.1 μM PH08 in 10% or 0% FBS for 15 min. Alternatively, cells were incubated at 4° C. for 30 min. Cells were then imaged under the epifluorescence NIR microscope. (Top) Representative fluorescence images of cells are shown. The contrast was normalized across all images. Scale bar=100 μm. (Bottom) Quantitative measurements of the fluorescence intensity in cells (n=3, mean #s.d.). *P<0.05 by one-way ANOVA followed by Tukey's multiple comparisons test.

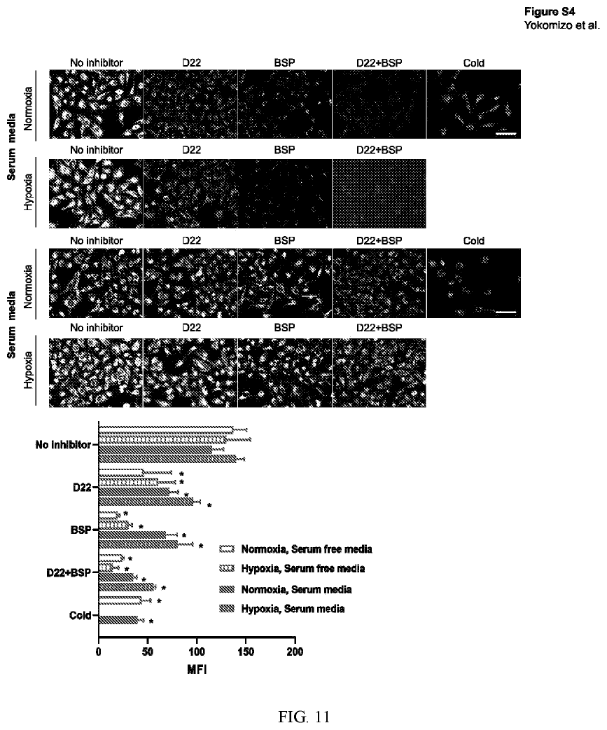

FIG. 12 In vivo evaluation of tumor cell targetability of pH sensing NIR fluorophores in a mouse model of ovarian cancer. (a) Intraoperative color and NIR fluorescence images of the abdominal cavity and peritoneal dissemination of ovarian cancer and major organs 4 h post-intraperitoneal injection of 20 nmol of PH03. Scale bar=5 mm. (b) Quantitative analysis of target-to-background ratio (TBR) of peritoneal tumors and major organs for PH03. TBR was determined by comparing the signals of tumors (Tu) or organs against muscle (Mu). Du, duodenum; He, heart; In, intestine; Ki, kidneys; Li, liver; Lu, lungs; Pa, pancreas; Sp, spleen; St, stomach (n=3, mean±s.e.m.). (c,d) Quantitative time-course assessment of TBR for up to 240 min post-injection of PH03 (n=3, mean±s.e.m.). (e) In vivo and ex vivo biodistribution of PH08 in major organs. 20 nmol of PH08 was injected intraperitoneally 4 h prior to imaging and resection. Abbreviations used are: Du, duodenum; He, heart; In, intestine; Ki, kidneys; Li, liver; Lu, lungs; Mu, muscle; Pa, pancreas; Sp, spleen; St, stomach; Tu, tumors. Scale bars=5 mm. (f) Intraoperative color and NIR fluorescence images of the abdominal cavity and peritoneal dissemination of human ovarian cancer SKOV3 4 h post-intraperitoneal injection of 20 nmol of PH08 in a xenograft model. Scale bar=5 mm. (g) Quantitative analysis of TBR of peritoneal SKOV3 tumors and major organs for PH08 (n=3, mean±s.e.m.). (h) Histological analysis of tumor-targeted PH08 in the orthotopic ovarian cancer model. Representative hematoxylin and eosin (H&E) and corresponding NIR fluorescence images are shown. Scale bars=50 μm.

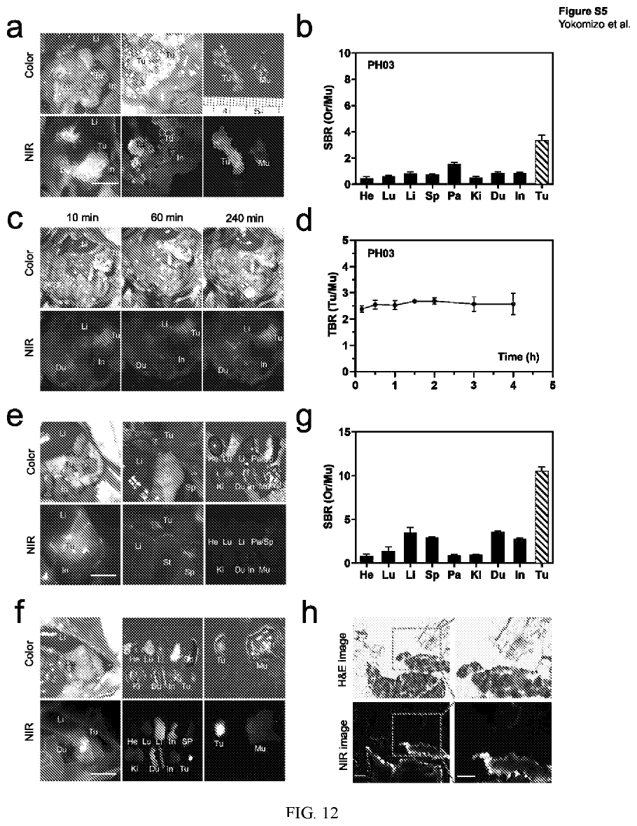

### DETAILED DESCRIPTION

Ovarian cancer is typically diagnosed at a late stage, which usually includes widespread peritoneal dissemination due to a lack of effective screening methods. Successful staging to map the localization of ovarian cancer and cytoreduction during surgery has been proven to significantly impact the prognosis of patients. One of the most significant prognostic factors is the amount of residual tumor after surgery. Therefore, it is crucial to reduce the tumor burden as much as possible, even when complete resection is impossible. However, accurate mapping and cytoreductive surgery for peritoneally-disseminated ovarian cancer, with numerous submillimeter lesions in multiple anatomical sites on the peritoneal surface, is often challenging and could lead to poor treatment outcomes and recurrence.

Fluorescence-guided surgery (FGS) aids surgeons with real-time visualization of small cancer foci and borders, which improves surgical and prognostic efficacy of cancer. Despite the steady advances in imaging devices, there is a scarcity of fluorophores available to achieve optimal FGS. FGS can be useful for ovarian cancer when a tumor-targeted fluorophore is used to localize small tumor lesions with sufficient specificity and sensitivity, resulting in successful surgical resection and minimal operating room time, and obviating the need for repeated operations. FGS has been approved for various procedures, including identifying tumor margin, sentinel lymph node mapping, angiography, and lymphography, and has improved tumor resection rates while minimizing normal tissue damage. Particularly, near-infrared (NIR) fluorescence imaging has led the field due to the reduced scattering, minimal tissue absorption, high tissue penetration depth, and low tissue autofluorescence interference, offering a high signal-to-background ratio (SBR) for deep tissue imaging.

In response to this, many tumor-targeted NIR probes have been actively developed for FGS. Particularly, “activatable” fluorophores hold promise in FGS by minimizing the background signal originating from non-target tissues and achieving elevated fluorescence signals upon targeting. “Always on” fluorescent probes usually require a considerably long time to clear from the background tissues to attenuate high background signal for targeted imaging, whereas activatable fluorescent probes typically show lower background signals resulting in higher tumor-to-background ratios (TBRs) upon activation and can recognize cancer-specific environments without washing out the unbound probes.

To this end, many rationally designed activatable fluorophores, especially those based on the concept of intramolecular photoinduced electron transfer (PeT), have been developed. In particular, acidic tissue microenvironments and intracellular compartments have been targeted for activatable probes. Typically, non-cancerous cells display an extracellular pH of around 7.5, while tumor cells show 6.4-7.1. Since imaging probes generally lack endogenous targeting mechanisms, they have been combined with a targeting moiety to enhance contrast, such as antibodies which are generally large in molecular weight and show slow clearance. This strategy results in a long wait time between administration and imaging (up to days) before a sufficiently high SBR is obtained, making this approach less attractive for FGS. In contrast, small molecules with molecular weights 10-1000 times less than peptides and proteins can distribute rapidly to their targets with fast elimination of off-target molecules, which renders high SBR. In addition, this approach is suitable for their clinical translation with economical, large-scale, and reproducible production.

Accordingly, the present disclosure provides pH-sensitive near-infrared fluorophore compounds that, e.g., exhibit rapid signal changes in acidic tumor microenvironments (TME) and mitochondrial and lysosomal retention. In one example, after topical application of the compound on peritoneal tumor regions in ovarian cancer-bearing mice, a rapid fluorescence increase (<10 min) and extended preservation of signals (>4 h post-topical application) can be observed, which together allow for the visualization of submillimeter tumors with a high tumor-to-background ratio (>5.0). Importantly, the compounds are permeable to cancer cells, e.g., via organic anion transporter peptides and colocalize in the mitochondria and lysosomes due to the positive charges, enabling a long retention time during FGS. As such, the compounds within the instant claims are advantageously useful in surgical and diagnostic applications.

### Compounds

In some embodiments, the present disclosure provides a compound of Formula (I):

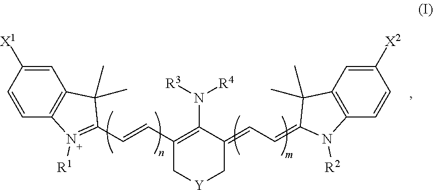

or a pharmaceutically acceptable salt thereof, wherein:
Y is selected from CH₂, O, and S;
X¹ and X² are each independently selected from H, halogen, CN, NO₂, OH, NH₂, C₁₋₃ alkyl, C₁₋₃ haloalkyl, C₁₋₃ alkoxy, C₁₋₃ haloalkoxy, C₁₋₃ alkylamino, and di(C₁₋₃ alkyl)amino;
n is 1 or 2;
m is 1 or 2;
R¹ and R² are each independently selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl;
R³ and R⁴ are each independently selected from H, C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl;
provided that at least one of R³ and R⁴ is other than H; or
R³ and R⁴ together with the N atom to which they are attached form a ring of formula:

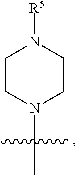

wherein R⁵ is selected from H, C₁₋₈ alkyl, C₁₋₈ haloalkyl, C(═O)Cy¹, and S(═O)₂Cy¹, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, or Cy¹; and
Cy¹ is selected from C₆₋₁₀ aryl and 5-10 membered heteroaryl, each of which is optionally substituted with 1 or 2 independently selected NO₂, CN, OH, C₁₋₃ alkoxy, C(═O)OH, NH₂, C₁₋₃ alkylamino, and di(C₁₋₃ alkyl)amino.

In some embodiments, n is 1. In some embodiments, n is 2. In some embodiments, m is 1. In some embodiments, m is 2. In some embodiments, n is 1 and m is 1. In some embodiments, n is 2 and m is 2. In some embodiments, n is 1 and m is 2. In some embodiments, n is 2 and m is 1.

In some embodiments, the compound has formula:

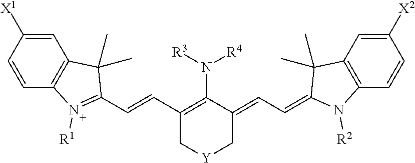

or a pharmaceutically acceptable salt thereof.

In some embodiments, Y is CH₂. In some embodiments, Y is O. In some embodiments, Y is S. In some embodiments, Y is selected from O and S.

In some embodiments, R¹ is C₁₋₈ alkyl. In some embodiments, R¹ is C₁₋₈ haloalkyl. In some embodiments, R¹ is C₁₋₈ alkyl, optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl. In some embodiments, R¹ is C₁₋₈ alkyl, optionally substituted with OH, C₁₋₃ alkoxy, C(═O)OH, SO₃H, P(═O)(OH)₂, NH₂, C₁₋₃ alkylamino, di(C₁₋₃ alkyl)amino, or tri(C₁₋₃ alkyl)amino. In some embodiments, R¹ is C₁₋₈ alkyl, optionally substituted with OH, C(═O)OH, SO₃H, or C₆₋₁₀ aryl. In some embodiments, R¹ is C₁₋₆ alkyl.

In some embodiments, R² is C₁₋₈ alkyl. In some embodiments, R² is C₁₋₈ haloalkyl. In some embodiments, R² is C₁₋₈ alkyl, optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl. In some embodiments, R² is C₁₋₈ alkyl, optionally substituted with OH, C₁₋₃ alkoxy, C(═O)OH, SO₃H, P(═O)(OH)₂, NH₂, C₁₋₃ alkylamino, di(C₁₋₃ alkyl)amino, or tri(C₁₋₃ alkyl)amino. In some embodiments, R² is C₁₋₈ alkyl, optionally substituted with OH, C(═O)OH, SO₃H, and C₆₋₁₀ aryl. In some embodiments, R² is C₁₋₆ alkyl.

In some embodiments, R¹ is C₁₋₈ alkyl, optionally substituted with OH, C(═O)OH, SO₃H, or C₆₋₁₀ aryl. In some embodiments, R² is C₁₋₈ alkyl, optionally substituted with OH, C(═O)OH, SO₃H, or C₆₋₁₀ aryl.

In some embodiments, X¹ is selected from H, halogen, CN, NO₂, OH, NH₂, C₁₋₃ alkyl, C₁₋₃ haloalkyl, C₁₋₃ alkoxy, C₁₋₃ haloalkoxy, C₁₋₃ alkylamino, and di(C₁₋₃ alkyl)amino. In some embodiments, X² is selected from H, halogen, CN, NO₂, OH, NH₂, C₁₋₃ alkyl, C₁₋₃ haloalkyl, C₁₋₃ alkoxy, C₁₋₃ haloalkoxy, C₁₋₃ alkylamino, and di(C₁₋₃ alkyl)amino.

In some embodiments, X¹ is H. In some embodiments, X² is H. In some embodiments, X¹ is H and X² is H. In some embodiments, X¹ is halogen. In some embodiments, X² is halogen. In some embodiments, X¹ is halogen and X² is halogen. In some embodiments, X¹ is H and X² is halogen. In some embodiments, X¹ is halogen and X² is H.

In some embodiments, R³ and R⁴ are each independently selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl.

In some embodiments, R³ and R⁴ are each independently selected from C₁₋₈ alkyl, optionally substituted with OH, C(═O)OH, SO₃H, or C₆₋₁₀ aryl (e.g., C₁₋₈ alkyl, optionally substituted with OH, or C₁₋₈ alkyl, optionally substituted with C(═O)OH or SO₃H). In some embodiments, R³ or R⁴ is C₁₋₈ alkyl, optionally substituted with OH, C(═O)OH, SO₃H, or C₆₋₁₀ aryl.

In some embodiments:
R³ is H, and R⁴ is selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl.

In some embodiments:
R⁴ is H, and R³ is selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl.

In some embodiments, R³ and R⁴ together with the N atom to which they are attached form a ring of formula:

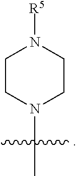

In some embodiments, the compound of Formula (I) is has formula:

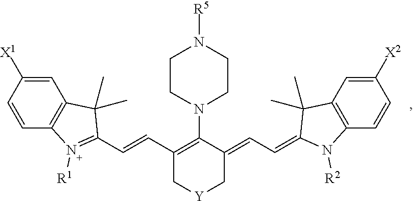

or a pharmaceutically acceptable salt thereof.

In some embodiments, R⁵ is selected from H, C₁₋₈ alkyl, C(═O)Cy¹, and S(═O)₂Cy¹, wherein said C₁₋₈ alkyl is optionally substituted with OH, C₁₋₃ alkoxy, C(═O)OH, SO₃H, and P(═O)(OH)₂.

In some embodiments, R⁵ is H. In some embodiments, R⁵ is selected from C₁₋₈ alkyl, C(═O)Cy¹, and S(═O)₂Cy¹, wherein said C₁₋₈ alkyl is optionally substituted with OH, C₁₋₃ alkoxy, C(═O)OH, SO₃H, and P(═O)(OH)₂. In some embodiments, R⁵ is C₁₋₈ alkyl, optionally substituted with OH, C₁₋₃ alkoxy, C(═O)OH, SO₃H, P(═O)(OH)₂, or Cy¹. In some embodiments, R⁵ is C₁₋₈ alkyl, substituted with OH, C₁₋₃ alkoxy, C(═O)OH, SO₃H, or P(═O)(OH)₂. In some embodiments, R⁵ is C₁₋₈ alkyl, substituted with Cy¹. In some embodiments, R⁵ is C(═O)Cy¹. In some embodiments, R⁵ is S(═O)₂Cy¹. In some embodiments, R⁵ is selected from H and S(═O)₂Cy¹.

In some embodiments, Cy¹ is selected from C₆₋₁₀ aryl and 5-10 membered heteroaryl, each of which is optionally substituted with 1 or 2 independently selected NO₂, CN, OH, C₁₋₃ alkoxy, C(═O)OH, NH₂, C₁₋₃ alkylamino, and di(C₁₋₃ alkyl)amino. In some embodiments, Cy¹ is C₆₋₁₀ aryl, optionally substituted with 1 or 2 independently selected NO₂, CN, OH, C₁₋₃ alkoxy, and C(═O)OH (e.g., with 2 NO₂ groups).

In some embodiments, Cy¹ is 5-10 membered heteroaryl, optionally substituted with 1 or 2 independently selected NO₂, CN, OH, C₁₋₃ alkoxy, and C(═O)OH.

In some embodiments, the compound of Formula (I) is selected from any one of the following compounds:

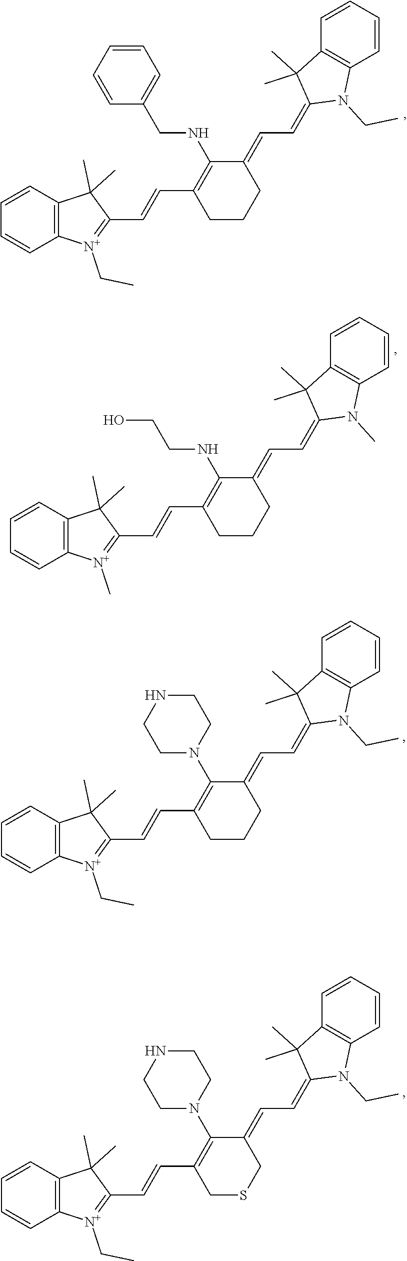 [SMILES: CCN1/C(=C/C=C2\CCCC(/C=C/C3=[N+](CC)c4ccccc4C3(C)C)=C2NCc2ccccc2)C(C)(C)c2ccccc21 ; CN1/C(=C/C=C2\CCCC(/C=C/C3=[N+](C)c4ccccc4C3(C)C)=C2NCCO)C(C)(C)c2ccccc21 ; CCN1/C(=C/C=C2\CCCC(/C=C/C3=[N+](CC)c4ccccc4C3(C)C)=C2N2CCNCC2)C(C)(C)c2ccccc21 ; CCN1/C(=C/C=C2\CSCC(/C=C/C3=[N+](CC)c4ccccc4C3(C)C)=C2N2CCNCC2)C(C)(C)c2ccccc21]

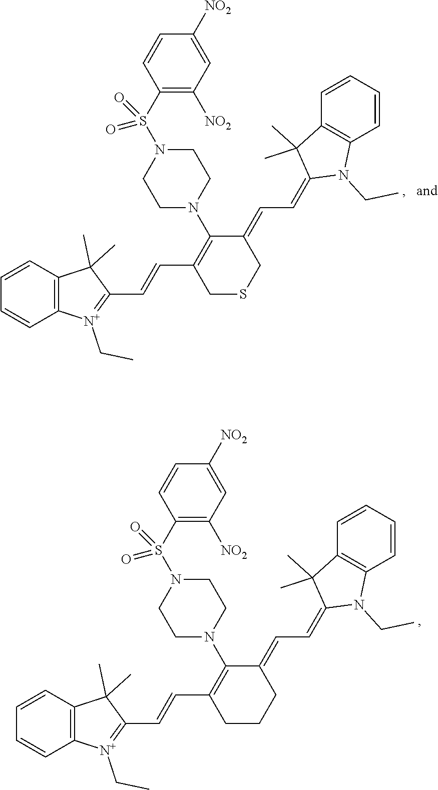 [SMILES: CCN1/C(=C/C=C2\CSCC(/C=C/C3=[N+](CC)c4ccccc4C3(C)C)=C2N2CCN(S(=O)(=O)c3ccc([N+](=O)[O-])cc3[N+](=O)[O-])CC2)C(C)(C)c2ccccc21 ; CCN1/C(=C/C=C2\CCCC(/C=C/C3=[N+](CC)c4ccccc4C3(C)C)=C2N2CCN(S(=O)(=O)c3ccc([N+](=O)[O-])cc3[N+](=O)[O-])CC2)C(C)(C)c2ccccc21]

or a pharmaceutically acceptable salt thereof.

Without being bound by any particular theory or speculation, it is believed that the compounds of Formula (I) possess fluorescent properties. In other words, the compounds are capable or emitting electromagnetic radiation or light of a wavelength.

In some embodiments, the radiation is visible (e.g., visible light) or invisible (e.g., ultraviolet, infrared, or near-infrared radiation). In some embodiments, the compounds are capable to absorbing light or radiation of a short wavelength and emitting light or radiation of a longer wavelength. In some embodiments, the emission maximum for the compounds of Formula (I) is within near-infrared or near-infrared II wavelength spectrum. In some embodiments, the emission maximum wavelength for the compound of Formula (I) is from about 600 nm to about 1800 nm, from about 600 to about 1000 nm, form about 650 to about 950 nm, from about 950 nm to about 1750 nm, from about 1000 to about 1700 nm, or from 1050 to about 1650 nm. In some embodiments, the emission maximum wavelength for the compound of Formula (I) is about 650 nm, about 700 nm, about 750 nm, about 800 nm, about 900 nm, about 950 nm, about 1000 nm, about 1050 nm, about 1100 nm, about 1150 nm, about 1200 nm, about 1250 nm, about 1300 nm, about 1500, about 1600 nm, about 1650 nm, about 1700 nm, or about 1750 nm. Without being bound by any particular theory of speculation, the emission maximum wavelength in NIR II window advantageously allows to use the compounds of Formula (I) for fluorescent imaging, because the compound's emitted NIR II radiation can be detected by a NIR-II surgical navigation system, a NIR-II camera, a NIR-II confocal/spinning-disc confocal microscope, a NIR-II light sheet microscope, NIR-II two-photon/multiphoton microscope, and NIR-II fluorescence lifetime microscope.

In some embodiments, the compound of Formula (I) is water-soluble. For example, aqueous solubility of the compound of Formula (I) is from about 1 g/L to about 250 g/L, or about 5 g/L, about 10 g/L, about 15 g/L, about 20 g/L, about 25 g/L, or about 100 g/L. Without being bound by any particular theory, aqueous solubility of the compound allows to obtain a sprayable aqueous solution of compound of Formula (I) for topical spraying application, for example, by using a spray-containing laparoscope for image guided surgery. During this surgery, a surgeon is able to spray the aqueous solution of the compound in the tumor microenvironment, thereby imaging the tumor. Suitable examples of tumors imageable using this method are described herein (e.g., ovarian, stomach, lung, or brain cancer). In some embodiments, a concentration of a compound of Formula (I) in the sprayable solution is from about 100 μM to about 1 mM.

### Pharmaceutically Acceptable Salts

In some embodiments, a salt of a compound of Formula (I) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of Formula (I) include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of Formula (I) include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compounds of Formula (I), or pharmaceutically acceptable salts thereof, are substantially isolated.

### Methods of Use

In one general aspect, the present application relates to compounds of formula (I) useful in imaging techniques, diagnosing and monitoring treatment of various diseases and conditions described herein. In some embodiments, because the compounds of Formula (I) can emit light after absorbing light, the compounds are useful in fluorescence imaging or optical imaging. In some embodiments, fluorescence imaging is NIR-II fluorescence imaging. In some embodiments, fluorescent imaging is carried out to detect light emitted by the compound of Formula (I) at a wavelength from about 950 nm to about 1750 nm, from about 1000 nm to about 1700 nm, about 1000 nm, about 1100 nm, about 1200 nm, about 1300 nm, about 1400 nm, about 1500 nm, about 1600 nm, or about 1700 nm.

Fluorescence imaging is a type of non-invasive imaging technique that can help visualize biological processes taking place in a living organism. This imaging technique is very sensitive, allowing to detect fluorophore-containing compounds in biological tissues even at picomolar concentration. The method commonly includes administering exogenously a fluorophore compound to a patient, then exciting the compound by pointing a source of exciting radiation (e.g. light) to a tissue where the compound is expected to be accumulated, the then detecting fluorescent emission at the tissue site.

In some embodiments, the present disclosure provides a method of imaging a tissue (e.g., cancerous tumor) in a subject, the method comprising (i) administering to the subject an effective amount of a compound of Formula (I), or a pharmaceutically acceptable salt thereof, or a pharmaceutical composition of comprising same, (ii) waiting a time sufficient to allow the compound to accumulate in the tissue (e.g., cancerous tumor) to be imaged; and (iii) imaging the tissue (e.g., cancerous tumor) with a fluorescence imaging technique. In some embodiments, the compound is administered systemically (e.g., using an injection and/or an injectable or infusible solution, e.g., as described herein). In some embodiments, the compound is administered topically (e.g., by topically spraying a composition, such as an aqueous solution, at the tissue containing a cancerous tumor).

In some embodiments, the tissue is selected from epithelial tissue, mucosal tissue, connective tissue, muscle tissue, skin tissue, fibrous tissue, vascular tissue, and nervous tissue. In some embodiments the is tissue is at or near an organ selected from lung, stomach, intestines, liver, thyroid, bladder, heart, eye, skin, kidney, gland, brain, pancreas, colon, lymph node, spleen, and prostate. In some embodiments, the issue is a cancerous tumor tissue. In some embodiments, the cancerous tumor tissue is selected from sarcoma, angiosarcoma, fibrosarcoma, rhabdomyosarcoma, liposarcoma, myxoma, rhabdomyoma, fibroma, lipoma, teratoma, lung cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

In some embodiments, the time sufficient to allow the compound of Formula (I) to accumulate in the cancerous tumor is from about 1 seconds to about 168 hours, from about 10 seconds to about 96 hours, from about 1 minute to about 96 hours, from about 48 hours to about 96 hours, from about 10 minutes to about 24 hours, from about 30 minutes to about 10 hours, about 10 minutes, about 15 minutes, about 30 minutes, about 1 hour, about 6 hours, about 24 hours, about 48 hours, about 72 hours, about 96 hours, about 120 hours, about 144 hours, or about 168 hours.

In some embodiments, the fluorescence imaging technique is NIR-II fluorescence imaging. The imaging can be carried out using a near infrared camera, imaging goggles, or telescope, or a similar device. In some embodiments, the device contains a source of light or irradiation to excite the fluorophore.

In some embodiments, the present disclosure provides a method of diagnosing (or early detection) of a disease or disorder (e.g., any of the cancers described herein). The method may include imaging the tissue (e.g., cancerous tissue) as described herein. For example, the method can include (i) administering to the subject an effective amount of a compound of Formula (I), or a pharmaceutically acceptable salt thereof, or a pharmaceutical composition comprising same; (ii) waiting a time sufficient to allow the compound to accumulate in the tissue (e.g., cancerous tumor), and (iii) imaging the tissue with an imaging technique.

In some embodiments, the present disclosure provides a method of treating cancer (any of the cancers described herein), the method comprising: (i) imaging a cancerous tumor in a subject according to an imaging method as described herein; and (ii) surgically removing the cancerous tumor from the subject. In some aspects of these embodiments, the present disclosure provides a method for in intraoperative optical and/or fluorescence imaging and image-guided cancer surgery. The imaging can be carried out as described herein, e.g., using a near infrared camera or vision goggles. Before imaging, the method may include administering a cancer-targeting fluorophore of Formula (I) and then waiting a sufficient amount of time (e.g., 1 min, 10 min, 30 min, 1 hour, 6 hours, 24 hours, 48 hours, 96 hour, or more) for the cancer-targeting compound to accumulate in the cancerous tissue. Suitable examples of cancer surgeries include staging surgery, tumor removal, debulking surgery, palliative surgery, reconstructive surgery, preventive surgery, laparoscopic surgery, laser surgery, cryosurgery, Mohs surgery, and endoscopy.

In some embodiments, the present disclosure provides a method of monitoring treatment of cancer in a subject, the method comprising (i) administering to the subject an effective amount of a compound of Formula (I), or a pharmaceutically acceptable salt thereof, or a pharmaceutical composition comprising same, (ii) waiting a time sufficient to allow the compound of Formula (I) to accumulate in cancerous tumor of the subject; (iii) imaging the cancerous tumor of the subject with an imaging technique; and (iv) administering to the subject a therapeutic agent in an effective amount to treat the cancer. In some embodiments, the method further includes step (v) after (iv), administering to the subject an effective amount of a compound of Formula (I), or a pharmaceutically acceptable salt thereof, or a pharmaceutical composition comprising same; (vi) waiting a time sufficient to allow the compound of Formula (I) to accumulate in the cancerous tumor of the subject; (vii) imaging the cancerous tumor of the subject with an imaging technique; and (viii) comparing the image of step (iii) and the image of step (vii). In one example, comparing the images is indicative of successful treatment of the cancer. Suitable examples of therapeutic agents useful to treat cancer include abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

### Compositions, Formulations, and Routes of Administration

The present application also provides pharmaceutical compositions comprising an effective amount of a compound of the present disclosure disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. The composition can be administered by any route by which a fluorophore is effectively administrable and that facilitates imaging of a tumorous tissue. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, the compound is administered systemically (e.g., intravascular or intravenous injection or infusion). In some embodiments, the compound is administered topically (e.g., in a sprayable solution).

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

Compositions, formulations, and dosage forms suitable for parenteral or for topical administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

### Dosages and Regimens

In the pharmaceutical compositions of the present application, a compound of the present disclosure is present in an effective amount (e.g., a therapeutically effective amount). Effective doses of the imageable compounds may vary, depending on the diseases treated, the severity of the disease, the route of administration, the sex, age and general health condition of the subject, excipient usage, the possibility of co-usage with other imaging agents or therapeutic treatments such as use of other agents and the judgment of the treating physician, lab technician, or a diagnostician.

In some embodiments, an effective amount of the compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound is about 0.1 mg/kg, about 0.5 mg/kg, about 1 mg/kg, about 2 mg/kg, or about 5 mg/kg.

The foregoing dosages can be administered as needed for imaging, for example, on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes kits useful, for example, in the imaging and/or treatment of disorders, diseases and conditions referred to herein, which include one or more containers containing a pharmaceutical composition comprising an effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit.

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula-O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula-NH₂.

As used herein, the term “Cₙ₋ₘ alkylamino” refers to a group of formula-NH (alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula-N (alkyl) 2, wherein the two alkyl groups each has, independently, n to m carbon atoms. In some embodiments, each alkyl group independently has 1 to 6, 1 to 4, or 1 to 3 carbon atoms.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphthyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the(S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, the disclosure provides a PEG (polyethylene glycol) derivative of a compound of Formula (I), wherein any H in the compound can be removed and replaced with a PEG moiety. Other exemplary embodiments are listed, for example, in commercial supplier catalogs, such as Shearwater Corporation's catalog “Polyethylene glycol and Derivatives for Biomedical Applications.” In some embodiments, the poly(ethylene glycol) molecule is a linear or a branched polymer. Linear or branched PEG can be alkylated (e.g., methylated or ethylated) at one termini. The molecular weight of the PEG may be between about 1,000 Da and about 100,000 Da. PEG can include repeating units of formula —(CH₂CH₂O)ₚ—, wherein 5 is an integer from 1 to 10,000.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

Chemicals and synthesis: All chemicals and solvents were of American Chemical Society grade or HPLC purity and were used as received. HPLC grade acetonitrile (CH₃CN) and water were purchased from VWR International (West Chester, PA) and American Bioanalytic (Natick, MA), respectively. All other chemicals were purchased from Fisher Scientific (Pittsburgh, PA) and Sigma-Aldrich (Saint Louis, MO), unless otherwise noted. Melting points were measured on a Meltemp apparatus and are uncorrected. ¹H- and ¹³C-NMR spectra were recorded on a BrukerAvance (400 MHZ) spectrometer. Vis/NIR absorption spectra were recorded on a Perkin Elmer Lambda 20 spectrophotometer or Varian 50 scan UV-visible spectrophotometer. Chemical purity was also confirmed using high-performance liquid chromatography (HPLC, Waters, Milford, MA) combined with simultaneous evaporative light scattering detection (ELSD), absorbance (photodiode array; PDA), fluorescence, and electrospray time-of-flight (ES-TOF) mass spectrometry (MS). High-resolution accurate mass spectra (HRMS) were obtained either using a Waters ESI-Q-TOF mass spectrometer or a Waters Micromass LCT TOF ES⁺ Premier Mass Spectrometer. All fluorophores were prepared as 10 mM stock solutions in dimethylsulfoxide (DMSO) prior to use.

Measurement of optophysical properties: Absorbance and fluorescence emission spectra of the series of NIR fluorophores were measured using fiber optic HR2000 absorbance (200-1100 nm) and USB2000FL fluorescence (350-1000 nm) spectrometers (Ocean Optics, Dunedin, FL). NIR excitation was provided by a 655 nm red laser pointer (Opcom Inc., Xiamen, China) set to 5 mW and coupled through a 300 μm core diameter, NA 0.22 fiber (Fiberguide Industries, Stirling, NJ). To determine the pH dependence of the fluorescence signal, optical measurements were performed at 37° C. in PBS, pH 4.0-7.4 or 100% FBS buffered with 50 mM HEPES, pH 4.0-7.4. The pH of the solutions was adjusted as appropriate with NaOH and HCl using a pH spear tester (Thermo Scientific). For fluorescence quantum yield (QY) measurements, indocyanine green (ICG) in DMSO (QY=13%) was used as a calibration standard to measure the fluorescence QY under conditions of matched absorbance at 765 nm. In silico calculations of the partition coefficient (LogP and LogD at pH 7.4), surface molecular charge, hydrophobicity, hydrogen bond acceptors/donors (HBA/HBD), the acid dissociation constant (pKa), and topological polar surface area (TPSA) were calculated using Marvin and JChem calculator plugins (ChemAxon, Budapest, Hungary). Theoretical calculations of HOMO and LUMO energy levels of fluorophores were performed based on density functional theory (DFT) with a 6-311 G basis set using Spartan 18 software (Wavefunction, Irvine, CA).

Live-cell labeling and in vitro imaging: Murine ovarian cancer cells (ID8 cells) were obtained from Dr. Wang's lab in the Vincent Center for Reproductive Biology at Massachusetts General Hospital (MGH). Cells were cultured and maintained in Dulbecco's modified Eagle's medium (DMEM) supplemented with 4% FBS (Gibco, Grand Island, NY), 1% insulin-transferrin-selenium (Sigma-Aldrich), and 1% penicillin-streptomycin (Gibco). Cells were plated in a T-75 culture flask until 70% confluency before dividing for the next passage. For fluorophore binding affinity, ID8 cells were incubated at a density of 20,000 cells/well in a 24-well plate in a humidified incubator at 37° C. under 5% CO₂ in the air. Cells were then incubated at 37° C. for 30 min in the presence of 0.2 μM of PH03 or PH08 and phenol red-free Hank's balanced salt solution (HBSS) with or without 10% FBS. To determine the response of NIR fluorophores to hypoxic conditions, cells were also incubated at 37° C. in a humidified incubator under 21% O2 and 5% CO₂ (normoxia) or 1% O₂ and 5% CO₂ (hypoxia) conditions for 24 h. The hypoxic condition was created using a hypoxic chamber (Coy Laboratory Products) with 5% CO₂/95% N₂ (Airgas, Lynn, MA). After washing three times with HBSS, live-cell imaging was performed using BioTek Cytation 5 (Winooski, VT) and NanoenTek JuLI Stage Live-Cell Imaging System (Seoul, S. Korea) with a Cy⁷ filter set (Chroma Technology, Brattleboro, VT). Image acquisition and analysis were performed using IPLab software (Scanalytics, Fairfax, VA), and the fluorescent intensity of each cell was measured using ImageJ (National Institutes of Health, Bethesda, MD). The outline of each cell was determined on a phase-contrast image to specify the region of interest (ROI) and superimposed on a fluorescence image for measurements. All NIR fluorescence images for each fluorophore were normalized identically for all conditions during an experiment. To determine the subcellular localization of NIR fluorophores, cells were seeded on a glass-bottomed dish (μ-Dish 35 mm low; Ibidi, Gräfelfing, Germany) at a density of 10,000 cells per dish and allowed to adhere overnight and then incubated in growth media with the presence of PH03 or PH08 at a concentration of 10 μM for 30 min. After being washed three times with HBSS, nuclei, mitochondria, and lysosomes were then stained with NucBlue (2 drops/mL), MitoTracker Green FM (0.5 μM) or Lyso Tracker Green (0.5 μM) for 20-30 min as per the manufacturer's instruction (Thermo Scientific, Waltham, MA). Cells were then washed with HBSS and imaged using the 4-channel Nikon TE2000 epifluorescence microscope.

Intraoperative NIR imaging: Seven-week-old female C57BL/6 mice were purchased from the Jackson Laboratory (Bar Harbor, ME) and housed in an AAALAC-certified facility at Massachusetts General Hospital (MGH). All animal procedures were performed in accordance with the Public Health Service Policy on Humane Care of Laboratory Animals and approved by the MGH IACUC (protocol #2016N000136). To establish a murine model of peritoneal dissemination of ovarian cancer, mice were injected intraperitoneally with 5×10⁶ cells in 100 μL saline. In this model, macroscopic metastatic intraperitoneal nodules appear reproducibly at approximately 90 d. To perform NIR fluorescence imaging, mice were fed chlorophyll-free mouse chow (VWR International) at least 5 d prior to imaging to minimize autofluorescence. For in vivo imaging of ovarian cancer, animals were anesthetized with 100 mg/kg ketamine and 10 mg/kg xylazine subcutaneously (Webster Veterinary, Fort Devens, MA). Following anesthesia, a midline incision was made to expose the abdominal cavity. To evaluate in vivo tumor targetability of the NIR fluorophores, mice were intraperitoneally injected with 20 nmol of ovarian cancer-targeted NIR fluorophores dissolved in 100 μL saline containing 5 wt/v % BSA. The abdominal cavity was opened with a midline incision, and NIR images were obtained over 4 h of the exposed abdominal cavity. For NIR fluorescence imaging, we used a custom-built real-time intraoperative NIR imaging system as described previously. In this study, a 760 nm excitation was used at a fluence rate of 4 mW/cm², with white light (400-650 nm; 40,000 lux). Color and NIR fluorescence images were acquired simultaneously with custom software at rates up to 15 Hz over a 10 cm diameter field-of-view (FOV). Pseudo-colored lime green was used for NIR fluorescence in the color-NIR merged images. The imaging system was positioned at a distance of 9 inches from the surgical field. For all real-time intraoperative imaging, a standardized imaging protocol was used during and after the operation. General FOV (5 cm in dia.) was used to include the pancreas, head, duodenum, liver, and kidneys of a mouse, while closeup FOV (3.3 cm in dia.) included the pancreas, head, and duodenum. Color and NIR fluorescence images were taken simultaneously. Real-time fluorescence signal in tumors (Tu) and tumor-to-background ratio (TBR) compared to neighboring tissue was obtained over the period of imaging, and the fluorescence intensity was plotted to evaluate in vivo molecular biodistribution and clearance. All images were obtained using the same exposure time. Mice were euthanized 4 h post-injection with CO₂ inhalation, and tumor tissues and the major organs, including the heart, lung, liver, pancreas, spleen, kidney, duodenum, intestine, muscle, and adnexa, were removed for ex vivo imaging and histological examination. NIR signals from resected tissues were imaged using the FLARE imaging system. The TBR was calculated using the same formula, with I_{T} representing the intensity of the tumor tissue and I_{B} representing the signal intensity of the surrounding tissue over the imaging period.

$$TBR = \frac{{I}_{T}}{{I}_{B}}$$

Preclinical intraoperative fluorescence-guided surgery: We performed preclinical fluorescence-guided surgery (FGS) in mice with peritoneal dissemination of ID8 ovarian cancer using the FLARE imaging system. We injected 20 nmol of PH08 in 100 μL of 5% wt/v BSA/saline into the mouse intraperitoneally 4 h prior to the surgery. The abdominal cavity was opened with a midline incision under isoflurane inhalational anesthesia. The entire abdominal cavity was examined carefully by a surgeon using color and fluorescent imaging on the FLARE imaging system. Peritoneal dissemination was resected under white light assisted with the color and fluorescent images.

Histological analysis of tumor-targetability of the NIR fluorophore: To determine the tissue distribution of the NIR fluorophore, tumors and major organs were removed from ID8 ovarian cancer-bearing mice 4 h post-injection with 20 nmol of PH08 in 100 μL of 5% wt/v BSA/saline. The dissected tissues were trimmed and embedded in Tissue-Tek optimum cutting temperature (OCT) compound (Sakura Finetek, Torrance, CA) without a pre-fixation step, and the tissue block was frozen at −80° C. Ten-μm thick frozen sections were cut by a cryostat (Leica, Germany). The slides were subject to fluorescence analysis first, then stained for hematoxylin and eosin (H&E). Fluorescence and brightfield images were acquired on the 4-channel Nikon TE2000 epifluorescence microscope. Image acquisition and analysis were performed using IPLab software (Scanalytics, Fairfax, VA). A custom filter set (Chroma Technology, Brattleboro, VT) composed of a 650/45 nm excitation filter, a 685 nm dichroic mirror, and a 720/60 nm emission filter were used for imaging. Exposure times were adjusted to obtain a similar maximum fluorescence value for each fluorescence image. Brightfield images of H&E-stained slides from a matching field of view were also obtained.

Statistical analysis: Statistical analysis was carried out using a one-way ANOVA followed by Tukey's multiple comparisons test. P values less than 0.05 were considered significant: *P<0.05, **P<0.01, and ***P<0.001. Results were presented as mean±standard errors of the mean (s.e.m.) for all the image analyses on the FLARE system and fluorescence microscopy. Statistical analysis and curve fitting were performed using Microsoft Excel and Prism version 8 software (GraphPad, San Diego, CA).

### Example 1—Characterization of pH Sensing NIR Fluorophores

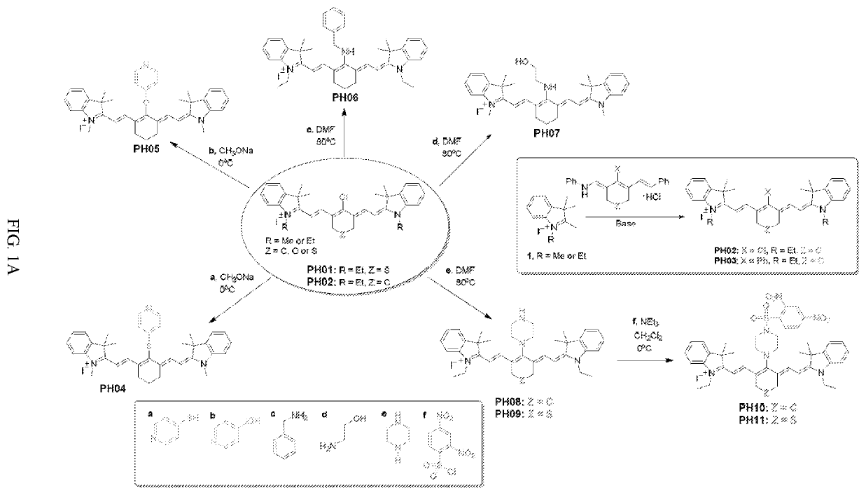

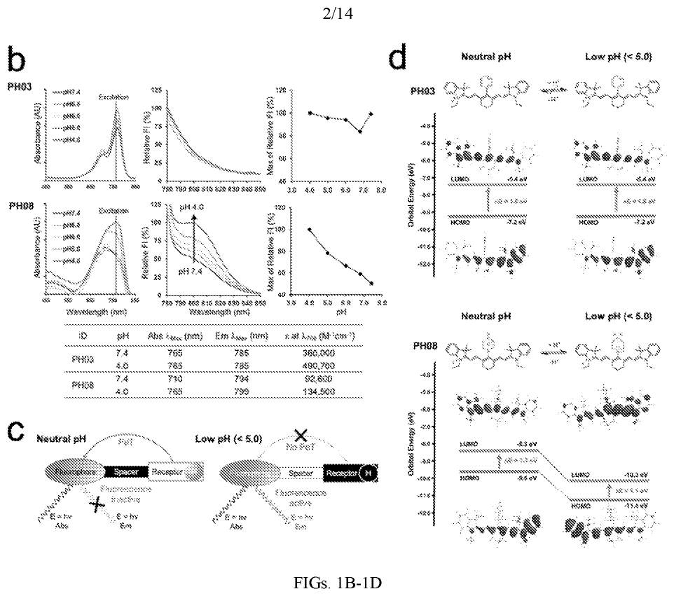

Systemic synthesis and optical characterization of pH sensing NIR fluorophores: To achieve rapid and durable intraoperative imaging, we rationally designed activatable NIR fluorophores to detect mildly low pH based on PeT by placing an amino group near the conjugated double bonds of a cyanine analog, which allows for the delocalization of electrons. As shown in FIG. 1a, the synthesis of heptamethine cyanines with a rigid cyclohexenyl ring in the polymethine backbone containing a reactive chlorine atom 2 (denoted as PH02) or a phenyl ring 3 (PH03) at the meso carbon was accomplished using our published synthetic methods. Then, the functionalized cyanine derivatives (PH04-PH08) were synthesized via S_{NR}1 reaction between the meso-chloride of PH02 and various nucleophiles such as aryl thioether, aryl ether, primary aryl amine, alkyl amine, and secondary amine, respectively, all suitable moieties to probe acidic environments. For pH-sensitive probes PH04 and PH05, due to the limited nucleophilicity of protonated thiol and hydroxyl groups, sodium methoxide was introduced as a base at a lower temperature to generate the reactive thiophenoxide and phenoxide ions. For the primary and secondary amine substitutions, due to the increased nucleophilic nature of the amines used, the synthesis of PH06-PH08 did not require the use of any bases. The crude products were purified by precipitation using DMSO/diethyl ether or methanol/diethyl ether or by column chromatography using 5-10% methanol in DCM as an eluent to produce PH04-PH08.

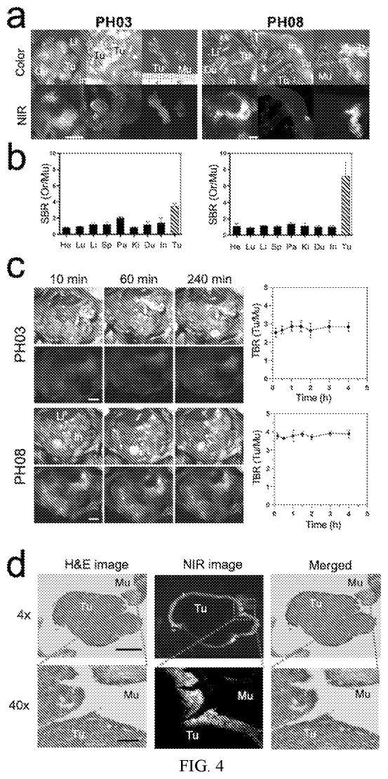

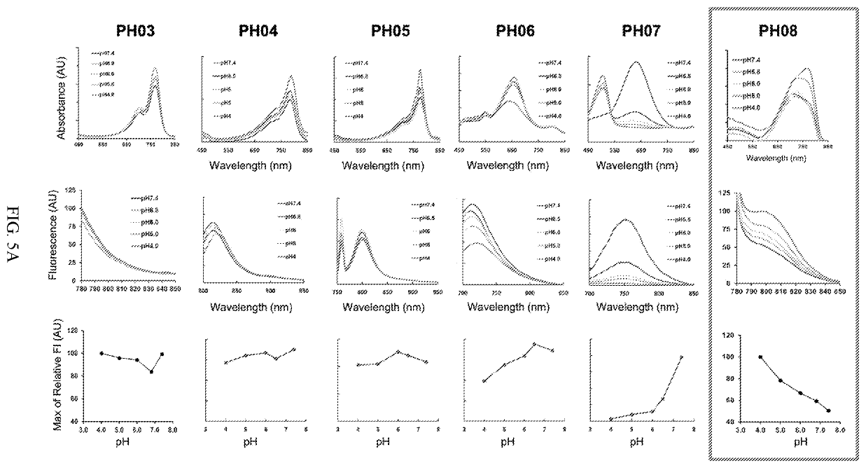

Upon measurement of the optical and physicochemical properties of the NIR fluorophores (Table 1), PH08 was found to be optically favorable for pH sensing of TME (FIG. 5). PH08 shows a modest molecular absorbance (& at 760 nm=92,600 M⁻¹ cm⁻¹) at neutral pH condition (pH=7.4) but increases in absorbance (ε at 760 nm=134,500 M⁻¹ cm⁻¹) accompanying with a red-shift from 710 to 765 nm when pH decreases to 4.0 (FIG. 1b). In contrast, PH03 displays little to no change in response to pH because it lacks the PeT mechanism (FIG. 1c). Based on these optical properties, we further examined the pH sensing properties of PH08 while using PH03 as a control. To understand the effect of PeT in response to pH changes, possible charge states, the optimized molecular structures, and frontier molecular orbitals (FMO) for compounds PH03 and PH08 in their deprotonated forms, PH08⁺ and PH08²⁺ were calculated based on DFT with 6-311 G basis set (FIG. 1d). PH03 and PH08 bear a phenyl or a piperazine moiety, respectively, at the central meso position, which significantly impacts the distribution of electrons within the highest occupied molecular orbitals (HOMOs) and the lowest unoccupied molecular orbitals (LUMOs) of these compounds. In the optimized molecular structures, the piperazine moiety of PH08 adopts a chair conformation in both the HOMO and LUMO energy levels. We further performed quantum chemical calculations on the protonated forms, PH08⁺ and PH08²⁺, using spartan 18. The energy gap decreases from 1.3 eV to 1.1 eV for PH08⁺ and PH08²⁺, which explains the absorption wavelength change of PH08 in different pH at 7.4 and 5.0. There are no distinct differences between the HOMO and LUMO for all the possible species of the optimized molecular structures. Based on the results of the optical property, PH08 advanced to further in vitro and in vivo testing for cancer imaging.

TABLE 1
Physicochemical and optochemical properties of pH sensing contrast agents.

| | MW | LogD, | TPSA | Rot. | Abs λ_{Max} | Em λ_{Max} | ε | QY | |
|---|---|---|---|---|---|---|---|---|---|
| ID | (Da) | pH 7.4 | (Å²) | bonds | (nm) | (nm) | (M⁻¹cm⁻¹) | (%) | TBR |
| PH03 | 553.81 | 6.78 | 6.25 | 6 | 765 | 785 | 364,800 | 21.7 | ++ |
| PH08 | 561.84 | 2.38 | 21.52 | 1 | 710 | 794 | 43,800 | 16.0 | +++ |

MW, molecular weight; TPSA, total polar surface area; Rot. Bonds, rotatable bonds; λ_{Max}, maximum wavelength; ε, extinction coefficient; QY, quantum yield. Tumor to background ratio (TBR) against normal pancreas was quantified and labeled as −, <1; +, 1-2; ++, 2-3; +++, 3-5. Measurements were performed at pH 7.4.

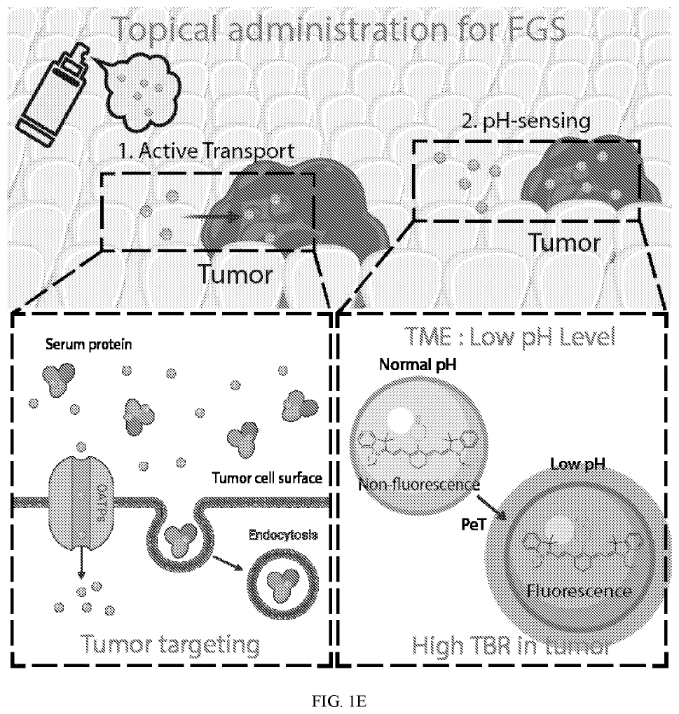

The role of organic anion transporters in the cellular uptake of NIR fluorophores: We next sought the mode of action for cellular uptake of the NIR fluorophores. Specifically, we hypothesized that the cellular uptake of heptamethine-fluorophores of PH08 in cancer tissues was mediated by membrane transporters. The cancer cell uptake of each fluorophore was examined in vitro using ID8 cells (a murine ovarian cancer cell line) by fluorescence microscopy. Both PH03 and PH08 accumulated in cells in a time-dependent manner (FIG. 2a,b).

Cellular uptake, however, significantly decreased when cells were incubated at 4° C., indicating that these fluorophores hardly enter cells via diffusion and that their entry is dependent on the action of transporters. The organic anion transporting polypeptides (OATPs) is known to mediate the transmembrane uptake of small molecule drugs and include structurally related fluorophores such as the clinically approved indocyanine green (ICG) and tumor-targeting IR-780. Interestingly, the cellular uptake was augmented under hypoxic conditions, which is consistent with the previous observation that OATPs have been established to be upregulated by hypoxia and expressed predominantly in cancer tissues compared with normal tissues. Next, we determined the contribution of organic cation transporters (OCTs) and organic carnitine (zwitterion) transporters (OCTNs) on the uptake of PH08, which may play roles in the transcellular movement of small organic molecules across epithelial barriers. To this end, ID8 ovarian cancer cells were treated with inhibitors for OATPs, OCTs, or OCTNs. The treatment with bromsulphthalein (BSP), which is an OATP inhibitor, resulted in a significant reduction in cellular uptake, while the treatment with D22, which is an inhibitor of OCTs and OCTNs, did not induce changes in cellular uptake (FIG. 2c,d), indicating that PH08 cellular uptake is mediated by OATPs.

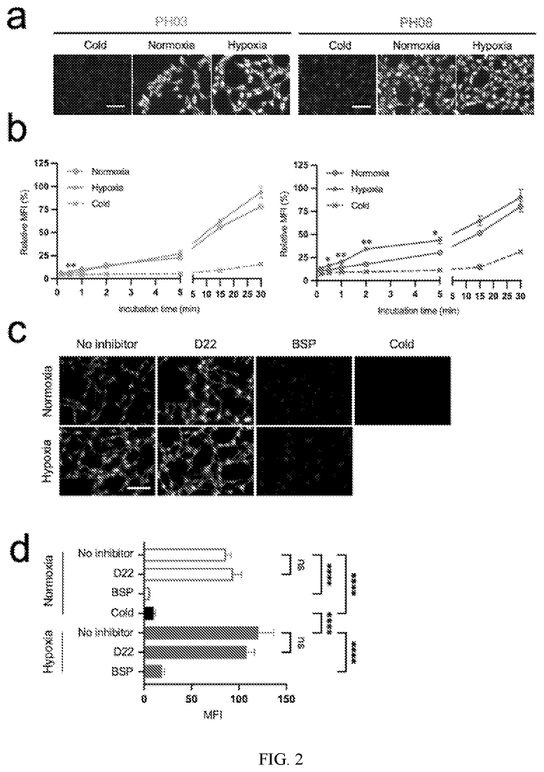

Subcellular distribution of pH sensing NIR fluorophores: To investigate the fate of the NIR fluorophores after uptake by cells, we performed in vitro live-cell imaging and evaluated subcellular localization of the NIR fluorophores in cultured ID8 ovarian cancer cells. PH03 and PH08 were incubated with ID8 cells for 30 min followed by double staining with commercially available MitoTracker or LysoTracker Green and imaged under the house-built NIR fluorescence microscope. NucBlue staining confirmed cell viability throughout the experiment. The fluorescence signal of PH03 overlapped with the signal of MitoTracker (91.47%), but significantly less with that of LysoTracker (34.93%) (FIG. 3a). On the other hand, PH08 showed much higher signals in the lysosome (71.83%), while maintaining similar signals in the mitochondria (83.05%) compared with PH03 (FIG. 3b). No signal was observed in nuclei for both NIR fluorophores. This is consistent with previous observations that lipophilic cations, including cyanine derivatives, accumulate in mitochondria and lysosomes. Together, these results suggest that PH08 enters cells via transporters and is well retained in cellular organelles.

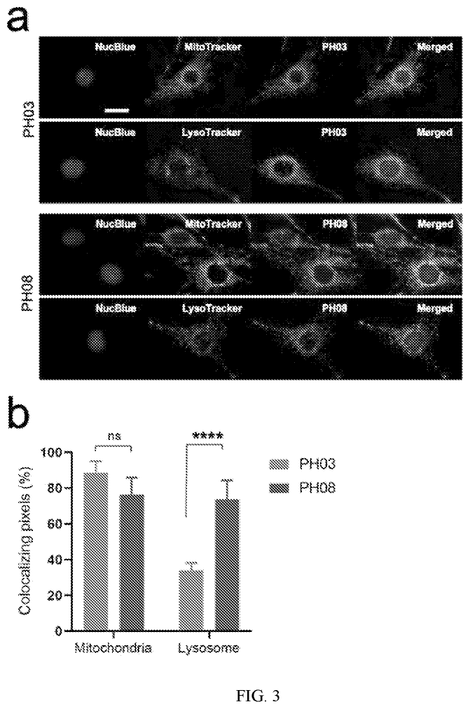

Evaluation of in vivo efficacy of NIR fluorophores for fluorescence-guided surgery (FGS): Having confirmed the pH sensitivity and tumor cell uptake of PH08 in vitro, we next evaluated the in vivo performance of the pH sensing NIR fluorophores in a mouse model of ovarian cancer. The intraperitoneal administration of an ID8 murine ovarian cancer cell line has been shown to closely mimic human advanced-stage ovarian cancer and its histology. In this model, approximately 12 week post-peritoneal injection of ID8 cells, numerous nodules were observed on the peritoneum. These small nodules (<1 mm) are extremely difficult to localize and visualize in the surgical field. Thus, this model was suitable to test the performance of this fluorophore. For imaging of ovarian cancer, 20 nmol of PH08 was administered intraperitoneally. The abdominal cavity was then imaged on the custom-built imaging system. Within 10 min of PH08 injection, small tumors planted on the peritoneal surface and omentum were clearly visualized (FIG. 4a) with a high TBR (˜6) against the surrounding normal tissues and other major organs due to ultralow background (FIG. 4b). The signal of PH08 allowed for clear visualization of the lesions that measured 5 mm or less than 1 mm in diameter. The TBR stayed high (>5.5) up to 4 h after injection (FIG. 4c,d). The pH insensitive NIR fluorophore PH03, with no PeT mechanism, also showed fluorescence signals in tumor tissue but displayed high signals in the normal tissue as well (FIG. 4a,b). Furthermore, due to the relatively high hydrophilicity of this fluorophore, the background signal remained high up to 4 h after administration with smaller TBRs (FIG. 4c). Having confirmed the in vivo efficacy of PH08, we performed FGS 4 h post-injection. The NIR imaging provided precise localization of peritoneal metastatic nodules and enabled the surgeon to achieve complete resection of metastatic nodules, including a small nodule measuring <1 mm in size.

In order to validate the tumor-specific fluorescence signal, frozen sections including both tumor and adjacent normal tissues harvested from tumor-bearing mice were examined by fluorescence microscopy. Histologically, the strong fluorescence signals of PH08 were detected throughout the tumor tissue with no substantial fluorescence signals in neighboring muscles, showing a clear contrast between tumors and surrounding normal tissues (FIG. 4d). The fluorescence signal was mostly seen on the top 3-4 layers of cells in the tumor tissue, of which the signal was found predominantly in the extracellular space and cytoplasm. There was a clear distinction in fluorescence intensity between cancerous and normal tissue, and no fluorescence signal was observed in the peritoneal surface nearby or in other surrounding normal tissues. These results suggest that the low molecular weight of PH08, along with its lipophilicity, supported efficient diffusion into tumor tissue, and its pH sensitivity helped achieve sufficiently high TBR for FGS. Together, these results suggest that this novel pH sensing NIR fluorophore is well suited for FGS and would merit advanced preclinical development.

In sum, we have successfully developed a pH-sensitive tumor-targeted contrast agent, PH08, which is rationally designed and functions via the PeT mechanism. In addition, PH08 is able to enter cancer cells via OATPs and shows excellent retention in the mitochondria and lysosomes. With these two mechanisms, PH08 can detect submillimeter peritoneal dissemination of ovarian cancer within 10 min of topical application with high TBR, and its specific fluorescence signal in tumor lesions was observed under NIR imaging up to 4 h. PH08 has overcome historical shortcomings of activatable NIR fluorophores, including long staining time, short retention time, and low TBR. PH08 shows favorable optical properties and increases its fluorescence in mildly acidic conditions between pH 7.0 and 4.0, contributing to its rapid staining of tumor tissue. Most pH sensing probes that have been developed to date only display PeT at relatively high pH values. Unlike other pH sensing probes, our rationally designed probe shows PeT in mildly acidic conditions, which favors specific detection of TME. A low molecular weight without the need for attachment of a targeting moiety is a critical property of PH08 because it enables local application, which passes through the acidic TME and highlights tumor margins with high TBR. These physicochemical properties had combinatorial effects on the TBR. This NIR fluorophore is therefore optimal for image-guided biopsy of superficial tumors, including colon and cervical cancer. Topical application of PH08 avoids systemic administration and unexpected off-target accumulation in vital organs, which reduces the risks of adverse effects. Moreover, this mode of administration generally requires a smaller dose compared to systemic application. In addition, since PH08 shows an excellent tumor targetability via OATPs and durable retention in organelles, its fluorescence signal is stable for at least 4 h after application. With this feature, it can also be used for laparoscopic or open surgery of peritoneal dissemination of ovarian and gastrointestinal cancer. Such an easy-to-use, economical and reliable tool is expected to have a significant impact on surgical care because image-guided robotic or laparoscopic surgery in the form of FGS is expected to be the predominant mode of surgery in the following decades.

Previously, other strategies have been used for the design of “activatable” probes to image tumor tissue. Generally, the activation process of these molecular probes requires hours to even days, which decreases the feasibility of this approach and chances for real-time imaging. The targeting of enzymatic activity, whose expression is elevated in tumor tissues, has the potential to achieve rapid detection of cancer. Cathepsin and MMP-activatable probes are based on the unquenching of stacked fluorophores; therefore, their fluorescent intensity upon activation is not sufficiently high to detect small cancer lesions. Topically applied activatable fluorophores are activated by the membrane-bound enzyme γ-glutamyl transpeptidase (GGT), which is overexpressed on the plasma membrane of tumor cells, enabling the fluorescent detection of small-sized tumors for up to 1 h post-injection. However, this method is still limited due to the complicated and large structure of the probe complex and lack of scalability for clinical use. PH08 with the PeT mechanism has the clear advantages of smaller molecular weight and simple chemical structure, which significantly reduces the production labor and cost.

The use of a small molecule as a contrast-enhancing agent has significant advantages in targeted delivery to tumor tissue because uptake of small molecules mainly relies on diffusion rather than convection. However, it has been challenging to obtain sufficiently high TBR using small molecule contrast-enhancing agents due to their rapid clearance, high background signal, and low target affinity. A small clinical trial shows the efficacy of an FDA-approved small molecule fluorophore ICG for FGS of ovarian cancer. In this study, due to the weak targetability of ICG, a high false-positive rate was reported. In the current study, both PH03 and PH08 were able to target ovarian cancer tissue and visualize the lesions (FIG. 4). Without an activation mechanism, however, “always-on” probes (e.g., PH03) rely on their targetability to specific tissues, which generally results in low TBR due to their elevated uptake in the background tissue unless they are designed for rapid renal clearance. In contrast, “activatable” PH08 with the same targeting mechanism was able to achieve high TBR due to the low background signal. This concept of using a “smart” small molecule with intrinsic targetability and activation mechanisms should be considered for the future design of molecular probes for cancer imaging and following FGS.

In conclusion, the rationally designed activatable PH08 in TME with PeT mechanisms and cancer cell permeability via OATPs shows rapid fluorescence recovery (<10 min) upon topical application and durable retention in the mitochondria and lysosomes of tumor cells (at least 4 h post-topical application). PH08 has the potential to reduce operation times and decrease the likelihood of residual tumors after surgery. Furthermore, since PH08 is a small molecule, it provides a scalable strategy for production and has a high potential for accelerated approval by the FDA for clinical use of tumor targeting, following the safety and regulatory guidelines of ICG. The rapidly acting pH-sensitive fluorophore, PH08, useful to highlight small tumor lesions with high TBR based on the PeT mechanism. PH08 enters cancer cells via organic anion transporter peptides (OATPs) and retains in the mitochondria and lysosomes, supporting a long retention time. The topical application of the rationally designed compounds of the invention, for example PH08, allows us to visualize small peritoneal dissemination within 10 min without washing, which lasts over 4 h post-administration, enabling real-time FGS.

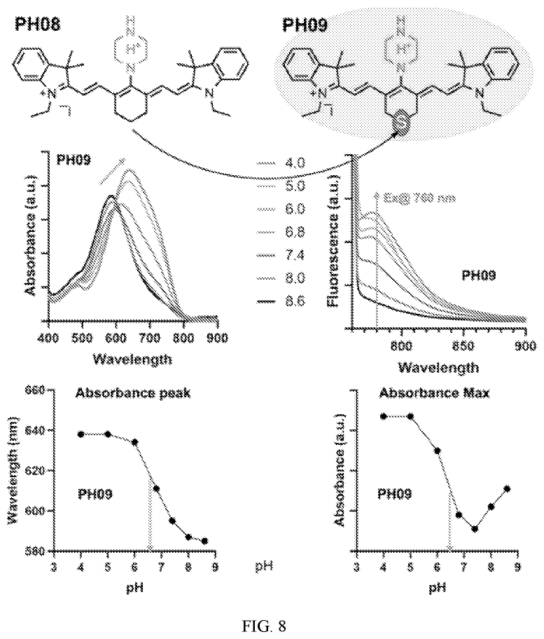

In addition, Sulfur-atom introduced PH01 was used to prepare PH09 and PH11, which can modulate dual emission ratio, i.e., ratiometric fluorescence imaging, in response to pH changes in the acidic tumor microenvironment and/or interactions with biological targets, including hypoxia and reactive oxygen species (FIG. 8).

### Examples 2—Preparation of Exemplified Compounds

As shown in FIG. 1a, the synthesis of heptamethine cyanines with a rigid cyclohexenyl ring in the polymethine backbone containing a reactive chlorine atom 2 or a phenyl ring 3 at the meso carbon was accomplished using our published synthetic method. Then, we followed our published procedure to synthesize the functionalized cyanine derivatives 4-8 via S_{NR}1 reaction between the meso-chloride of PH02 and various nucleophiles such as aryl thioether, aryl ether, primary aryl amine, alkyl amine, and secondary amine respectively, all suitable moieties to probe the acidic environments.

For pH sensitive probes PH04 and PH05, due to the limited nucleophilicity of protonated thiol and hydroxyl groups, sodium methoxide was introduced as a base at a lower temperature to generate the reactive thiophenoxide and phenoxide ions. Then, the more nucleophilic thiolate or phenoxide ion was allowed to react with dye 2 at room temperature to produce the pH-sensitive probes PH04 and PH05. For the primary and secondary amine substitutions, due to the increased nucleophilic nature of the amines used, the synthesis of pH probes 6-8 (PH06-08) did not require the use of any bases. Therefore, various amines can react with PH02 in DMF without any base at room temperature or through moderate heating of the reaction mixture to furnish the final probes. The crude products of FIG. 1a were purified by precipitation using DMSO/diethyl ether or methanol/diethyl ether or by column chromatography using 5-10% methanol in DCM as an eluent to produce the pH-sensitive PH04-08 in good yields

1,2,3,3-Tetramethyl-3H-indole-1-ium iodide salt (1; R=Me or Et) and 2-((E)-2-((E)-2-chloro-3-(2-((E)-1,3,3-trimethylinden-2-ylidene)ethylidene) cyclohex-1-en-1-yl) vinyl)-1,3,3-trimethyl-3H-indol-1-ium iodide (2; R=Me or Et) were prepared based on published protocol.

1-Ethyl-2-((E)-2-((E)-6-(2-((E)-1-ethyl-3,3-dimethylindolin-2-ylidene)ethylidene)-3,4,5,6-tetrahydro-[1,1′-biphenyl]-2-yl) vinyl)-3,3-dimethyl-3H-indol-1-ium iodide (3, R=Et) and 1,3,3-trimethyl-2-((E)-2-((E)-2-((E)-2-(pyridine-4-ylthio)-3-((E)-2-(1,3,3-trimethylindolin-2-ylidene)ethylidene) cyclohex-1-en-1-yl) vinyl)-3H-indol-1-ium iodide (4) were prepared based on published procedures.

1,3,3-trimethyl-2-((E)-2-((E)-2-((E)-2-(pyridine-4-yloxy)-3-((E)-2-(1,3,3-trimethylindolin-2-ylidene)ethylidene) cyclohex-1-en-1-yl) vinyl)-3H-indol-1-ium iodide (5),

2-((E)-2 ((E)-2-(benzylamino)-3-(2-((E)-1-ethyl-3,3-dimethylindolin-2-ylidene)ethylidene)-cyclohex-1-en-1-yl) vinyl)-1-ethyl-3,3-dimethyl-3H-1-indol-1-ium, iodide (6), and

2-((E)-2-((E)-2-((2-hydroxyethyl)amino)-3-(2-((Z)-1,1,3-trimethyl-1,3-dihydro-2H-inden-2-ylidene)ethylidene) cyclohex-1-en-1-yl) vinyl)-1,3,3-tri2-((E)-2-((E)-4-chloro-5-(2-((E)-1-ethyl-3,3-dimethylindolin-2-ylidene)ethylidene)-5,6-dihydro-2H-thiopyran-3-yl) vinyl)-1-ethyl-3,3-dimethyl-3H-indol-1-ium methyl-3H-indol-1-ium iodide (7)

were prepared according to the previously reported procedures.

2-((E)-2-((E)-3-(2-((E)-1-ethyl-3,3-dimethylindolin-2-ylidene)ethylidene)-2-(piperazin-1-yl)cyclohex-1-en-1-yl) vinyl)-3,3-dimethyl-3H-indol-1-ium iodide (8). Chlorocyanine dye 2 (860 mg, 1.5 mmol) and piperazine (480 mg, 6 mmol) were dissolved in anhydrous DMF (15 mL) and stirred at 80° C. for 2 h under a nitrogen atmosphere. The solvent was removed under reduced pressure, and the resulting residue was purified by silica gel chromatography using methanol/CH₂Cl₂ as an eluent to give a greenish blue solid (800 mg, 80%). Mp 167-169° C. ¹H NMR (400 MHz, DMSO-d₆) 1.25 (t, 6H, J=7.0 Hz), 1.63 (s, 12H), 1.73-1.81 (m, 2H), 2.51-2.56 (m, 4H), 3.09-3.14 (m, 4H), 3.68-3.73 (m, 4H), 4.10 (q, 4H, J=7.0 Hz), 5.95 (d, 2H, J=13.4 Hz), 7.12-7.18 (m, 2H), 7.23 (d, 2H, J=7.8 Hz), 7.33-7.39 (m, 2H), 7.53 (d, 2H, J=7.3 Hz), 7.61 (d, 2H, J=13.4 Hz). ¹³C NMR (400 MHZ, DMSO-d₆): 12.1, 21.8, 25.0, 28.7, 38.3, 46.9, 48.1, 54.7, 96.2, 110.1, 122.7, 123.6, 124.1, 128.8, 140.6, 140.7, 142.6, 168.2. LC MS (m z): 560.9 (M⁺)

2-((E)-2-((E)-2-(4-((2,4-dinitrophenyl) sulfonyl) piperazin-1-yl)-3-(2-((E)-1-ethyl-3,3-dimethylindolin-2-ylidene)ethylidene) cyclohex-1-en-1-yl) vinyl)-1-ethyl-3,3-dimethyl-1-indol-1-ium, iodide (9). To an anhydrous solution of compound 8 (230 mg, 0.40 mmol) in dichloromethane and acetone (1:1), 2,4-dinitrobenzenesulfonyl chloride (1.2 equiv.) was added portion wise at 0° C. in the presence of a catalytic amount of triethylamine under a nitrogen atmosphere. After completion of the reaction as confirmed by TLC and UV, the reaction mixture was cooled to room temperature and the solvents were removed under reduced pressure. The crude product was purified by silica gel column chromatography eluted with CH₂Cl₂/CH₃OH (9:1, v/v). The pure product was obtained in an 80% yield. Mp: 219-222° C. ¹H NMR (400 MHZ, DMSO-d₆): 1.26 (t, 6H, J=6.5 Hz), 1.51 (s, 12H), 1.71-1.79 (m, 2H), 3.49-3.53 (m, 4H), 3.59-3.69 (m, 4H), 4.08-4.19 (m, 4H), 6.05 (d, 2H, J=13.5 Hz), 7.17-7.23 (m, 2H), 7.31-7.41 (m, 4H), 7.50 (d, 2H, J=13.5 Hz), 8.42 (d, 1H, J=8.8 Hz), 7.73-7.83 (dd, 1H, J=2.5 Hz, 8.8 Hz), 9.28 (d, 1H, J=2.5 Hz). ¹³C NMR (400 MHZ, DMSO-d₆): 12.4, 25.1, 28.1, 30.9, 48.6, 50.9, 53.4, 87.8, 110.8, 122.8, 125.8, 141.1, 141.9, 142.2, 148.5, 151.2, 169.7. LC MS (m z): 790 (M⁺−1).

Additional details of exemplified compound preparation are provided below.

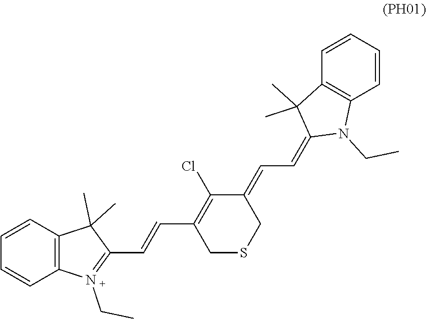

### 2-((E)-2-((E)-4-chloro-5-(2-((E)-1-ethyl-3,3-dimethylindolin-2-ylidene)ethylidene)-5,6-dihydro-2H-thiopyran-3-yl) vinyl)-1-ethyl-3,3-dimethyl-3H-indol-1-ium

The synthesis of heptamethine cyanine fluorophore PH01 was achieved by the condensation reaction between the heterocyclic 3H-indolium salts 1 (R=Et) (2 mmol) and modified Vilsmeier-Haack reagent with sulfur atom (1 mmol) under a nitrogen atmosphere was heated under reflux for 1 h. Removal of ethanol on a rotary evaporator was followed by treatment of the residue with chloroform, and then filtration of the solution from sodium acetate. Chromatography on silica gel (methanol/chloroform, 1:19) was followed by crystallization from methanol/ether to give (60%) of PH01 iodide. Yield (60%, 0.248 g); ¹H NMR (400 MHZ, CDCl₃): δ ppm 1.48 (t, J=6.29, 6H), 1.73 (S, 12H), 3.86 (S, 4H), 4.34-4.36 (m, 4H), 6.31 (d, J=14.21 2H), 7.23-7.31 (m, 4H), 7.41-7.43 (m, 4H), 8.42 (d, J=14.21, 2H), 13C NMR (100 MHz, CDCl₃): δ ppm 12.68, 28.04, 28.55, 40.43, 49.60, 101.14, 141.29, 141.60, 144.67, 150.63, 172.97 λabs=775 nm in MeOH.

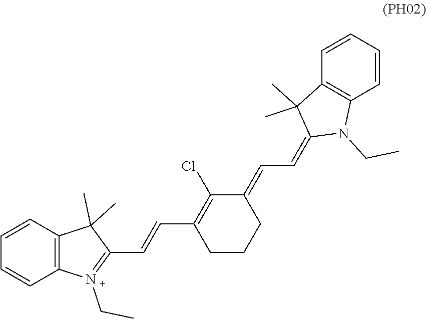

### 2-[4′-Chloro-7′-(l″-alkyl-3″,3″-dimethylindolin-2″-ylidene)-3′,5′-(propane-r″,3″,-diyl)-r,3 l,5,-heptatrien-r-yl]-1-alkyl-3,3-dimethylindolinium iodide (alkyl=Me or Et)

A solution of 1-alkyl-2,3,3-trimethylindolinium iodide 1 (630 mg, 2 mmol) (R=Et), and N-[5-anilino-3-chloro-2,4-(propane˜r,3′-diyl)-2,4-pentadien-1-ylidene]anilinium chloride (360 mg, 1 mmol), and anhydrous sodium acetate (200 mg) under a nitrogen atmosphere was heated under reflux for 1 h. Removal of ethanol on a rotary evaporator was followed by treatment of the residue with chloroform, and then filtration of the solution from sodium acetate. Chromatography on silica gel (methanol/chloroform, 1:19) was followed by crystallization from methanol/ether to give 518 mg (81%) of PH02 iodide.

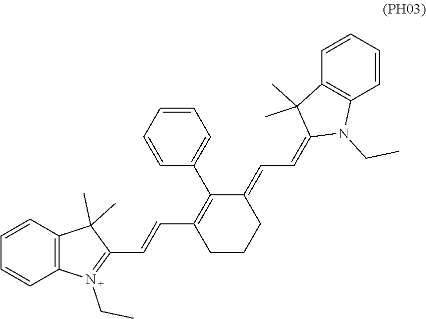

### 1-Ethyl-2-((E)-2-((E)-6-(2-((E)-1-ethyl-3,3-dimethylindolin-2-ylidene)ethylidene)-3,4,5,6-tetrahydro-[1,1′-biphenyl]-2-yl) vinyl)-3,3-dimethyl-3H-indol-1-ium iodide (R=Et)

A solution of POCl₃ (11 mL, 117.66 mmol) in dichloromethane (10 mL) was added dropwise to a solution of DMF (13 mL, 167.89 mmol) indichloromethane (13 mL) at 0° C. for 30 min under inert conditions. Then 1-phenylcyclohexene (5.5 mL, 32.81 mmol) was dissolved in dry dichloromethane (5 mL) and added dropwise to the solution which was then refluxed for 3 h. The solution was allowed to cool to room temperature and then poured over 500 mL of ice/water. Aniline (9 mL, 98.57 mmol) in ethanol (9 mL) was added to cap the ends. The crude solid was collected and washed with diethyl ether and hexanes. Resulting in the phenyl modified dianil linker as a pure compound and used without further purification. The ethyl indolium 1 (2 molar eq), the phenyl modified dianil linker (1 molar eq), and sodium acetate (2 molar eq) were dissolved in acetic anhydride and heated to 60° C. for 2-3 h. The crude product was then precipitated with diethyl ether, collected, and washed with methanol to yield heptamethine PH03 as pure sample. Yield: 70%; m.p. >260° C.; ¹H NMR (acetone-d₆) δ 1.11 (s, 12H), 1.24 (t, J=7.2 Hz, 6H), 1.96 (m, 2H), 2.97 (t, 4H), 4.14 (m, 4H) 6.20 (d, J=14 Hz, 2H), 7.18 (m, 4H), 7.35 (m, 4H), 7.47 (d, J=7.6 Hz, 2H), 7.61 (m, 3H); ¹³C NMR (acetone-d₆) δ 12.5, 21.3, 24.6, 27.4, 39.8, 40.0, 40.2, 48.7, 100.2, 111.2, 123.0, 125.0, 128.6, 129.0, 129.1, 129.6, 131.1, 141.1, 142.1, 147.7, 171.2. HRMS (ESI) m/z: calcd. for C₄₀H₄₅N⁺ 553.3577, found 553.1566.

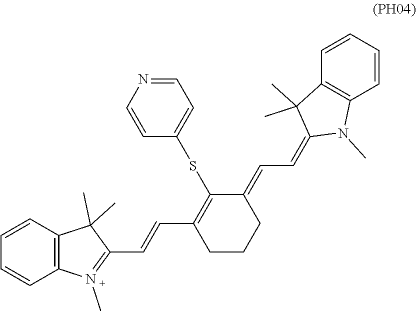

### 1,3,3-trimethyl-2-((E)-2-((E)-2-((E)-2-(pyridine-4-ylthio)-3-((E)-2-(1,3,3-trimethylindolin-2-ylidene)ethylidene) cyclohex-1-en-1-yl) vinyl)-3H-indol-1-iumiodide

Pyridine-4-thiol (1 mmol) and sodium methoxide (1 mmol) in DMF were stirred in an ice bath for 30 min under nitrogen atmosphere. PH02 (1 mmol) was dissolved in DMF (5 mL) and added dropwise into the flask. The reaction was stirred at room temperature for 2 h and reaction progress was monitored by UV-vis spectroscopy. After the reaction had completed, diethyl ether was added to precipitate the product. The crude compound was recrystallized in methanol/diethyl ether to obtain pure dye (208 mg, 71%). Mp: >260° C. Vis/NIR abs, λmax: 807 nm. ¹H NMR (400 MHZ, CDCl₃) δ 1.47 (s, 12H), 2.07-2.10 (m, 2H), 2.90 (s, 4H), 3.77 (s, 6H), 6.26 (d, J=14.0 Hz, 2H), 7.12-7.16 (m, 4H), 7.21-7.25 (m, 2H), 7.28-7.30 (m, 4H), 7.39 (t, J=8.0 Hz, 2H), 8.41-8.42 (m, 2H), 8.53 (d, J=14.0 Hz, 2H). ¹³C NMR (100 MHz, CDCl₃) δ 20.6, 26.9, 27.7, 32.7, 49.1, 102.5, 110.8, 120.3, 122.0, 125.4, 128.8, 134.5, 140.9, 142.6, 145.1, 148.4, 149.9, 172.9.

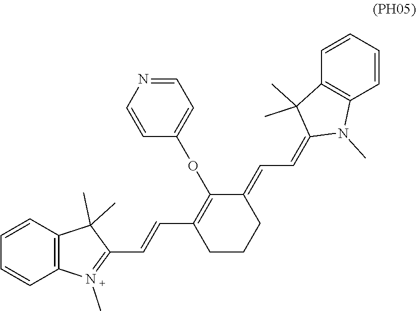

### 1,3,3-trimethyl-2-((E)-2-((E)-2-((E)-2-(pyridine-4-yloxy)-3-((E)-2-(1,3,3-trimethylindolin-2-ylidene)ethylidene) cyclohex-1-en-1-yl) vinyl)-3H-indol-1-ium iodide

Pyridin-4-ol (1 mmol) and sodium methoxide (1 mmol) in DMF were stirred in an ice bath for 30 min under a nitrogen atmosphere. PH02-R=Me (1 mmol) was dissolved in DMF (5 mL) and added dropwise into the flask. The reaction mixture was stirred at room temperature for 2 h, and reaction progress was monitored by UV-vis spectroscopy. After the reaction had completed, diethyl ether was added to the mixture to precipitate the product. The crude compound was recrystallized in methanol/diethyl ether to obtain pure dye (140 mg, 25%). Mp: >260° C. Vis/NIR abs, λmax: 786 nm. ¹H NMR (400 MHZ, DMSO) δ 1.40 (s, 12H), 1.93 (s, 2H), 2.73 (s, 4H), 3.67 (s, 6H), 6.32-6.38 (m, 4H), 7.08 (d, J=14.0 Hz, 2H), 7.25-7.27 (m, 2H), 7.41-7.43 (m, 4H), 7.58 (d, J=7.6 Hz, 2H), 7.73 (d, J=7.2 Hz, 2H). ¹³C NMR (100 MHz, DMSO) δ 24.4, 27.6, 32.0, 49.2, 102.6, 118.1, 122.9, 125.7, 126.7, 129.0, 140.7, 141.5, 142.2, 143.1, 153.0, 172.9, 177.2.

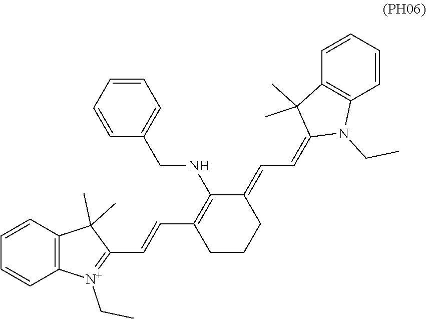 [SMILES: CCN1/C(=C/C=C2\CCCC(/C=C/C3=[N+](CC)c4ccccc4C3(C)C)=C2NCc2ccccc2)C(C)(C)c2ccccc21]

### 2-((E)-2 ((E)-2-(benzylamino)-3-(2-((E)-1-ethyl-3,3-dimethylindolin-2-ylidene)-ethylidene)-cyclohex-1-en-1-yl) vinyl)-1-ethyl-3,3-dimethyl-3H-1-indol-1-ium, iodide

To a solution of chlorocyanine PH02-R=Et-(315 mg, 0.5 mmol) in anhydrous acetonitrile was added benzylamine (4 equiv) in the presence of 0.1 equiv of N,N-diisopropylethylamine. The reaction mixture was stirred at 80° C. for 3 h, after completion of the reaction the solvent was removed under reduced pressure, and the resulting residue was purified by the silica gel chromatography using methanol/CH₂Cl₂ as eluent to give a blue solid in 70% yield. Mp. 159-161° C. ¹H NMR (400 MHz, DMSO-d₆): δ 1.21 (t, J=6.5 Hz, 6H), 1.37 (s, 12H), 1.76-1.79 (m, 2H), 2.49-2.58 (m, 4H), 3.88-3.91 (m, 4H), 4.91 (s, 2H), 5.80 (d, J=14.2 Hz, 2H), 7.03-7.12 (m, 2H), 7.12-7.16 (d, J=8.5 Hz, 2H,), 7.35-7.54 (m, 12H), 7.59 (d, J=14.2 Hz, 2H,), 8.21 (brs, 1H). ¹³C NMR (400 MHZ, DMSO-d₆): δ 11.7, 21.6, 25.5, 28.1, 42.8, 47.4, 53.0, 94.6, 109.3, 122.4, 122.9, 127.4, 129.0, 129.1, 129.3, 129.4, 134.4, 138.7, 140.3, 142.8, 145.8, 166.9.

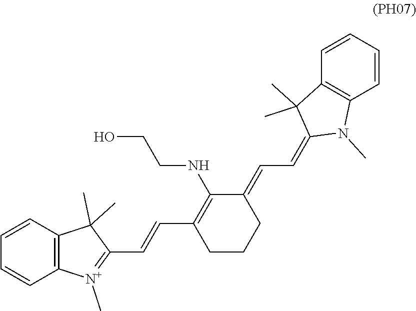 [SMILES: CN1/C(=C/C=C2\CCCC(/C=C/C3=[N+](C)c4ccccc4C3(C)C)=C2NCCO)C(C)(C)c2ccccc21]

### 2-((E)-2-((E)-2-((2-hydroxyethyl)amino)-3-(2-((Z)-1,1,3-trimethyl-1,3-dihydro-2H-inden-2-ylidene)ethylidene) cyclohex-1-en-1-yl) vinyl)-1,3,3-trimethyl-3H-indol-1-ium iodide

PH02 (1.0 mmol) and 2-aminoethan-1-ol (4 mmol) in DMF (5 mL) were heated at room temperature to 85° C. under a nitrogen atmosphere and reaction progress was monitored by UV-vis spectroscopy. Upon completion, the solvent was removed under reduced pressure. The crude product was purified by silica gel chromatography column in MeOH/DCM (5%) to afford blue solid (30 mg, 30%). Mp: 185-187° C. Vis/NIR abs, λmax: 633 nm. ¹H NMR (400 MHZ, CDCl₃): δ 1.67 (s, 12H), 1.84-1.87 (m, 2H), 2.49-2.52 (m, 4H), 3.42 (s, 6H), 3.99 (s, 4H), 5.58 (m, 2H), 6.90-6.92 (m, 2H), 7.07-7.11 (m, 2H), 7.28-7.31 (m, 4H), 7.84 (m, 2H). ¹³C NMR (100 MHz, DMSO): δ 21.34, 24.68, 28.15, 47.08, 52.17, 60.47, 94.45, 108.09, 121.97, 122.34, 128.06, 138.25, 139.73, 143.51, 167.63.

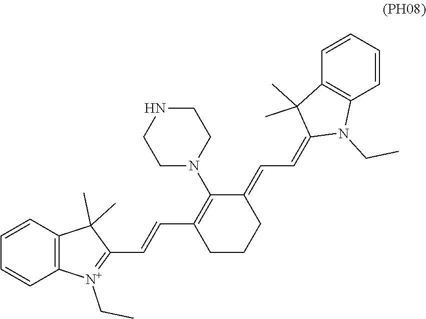 [SMILES: CCN1/C(=C/C=C2\CCCC(/C=C/C3=[N+](CC)c4ccccc4C3(C)C)=C2N2CCNCC2)C(C)(C)c2ccccc21]

### 2-((E)-2-((E)-3-(2-((E)-1-ethyl-3,3-dimethylindolin-2-ylidene)ethylidene)-2-(piperazin-1-yl)cyclohex-1-en-1-yl) vinyl)-3,3-dimethyl-3H-indol-1-ium iodide

PH02 (860 mg, 1.5 mmol) and piperazine (480 mg, 6 mmol) were dissolved in anhydrous DMF (15 mL) and stirred at 80° C. for 2 h under a nitrogen atmosphere. The solvent was removed under reduced pressure, and the resulting residue was purified by silica gel chromatography using methanol/CH₂Cl₂ as an eluent to give a greenish blue solid (800 mg, 80%). Mp 167-169° C. ¹H NMR. (400 MHZ, DMSO-d₆) δ 1.25 (t, 6H, J=7.0 Hz), 1.63 (s, 12H), 1.73-1.81 (m, 2H), 2.51-2.56 (m, 4H), 3.09-3.14 (m, 4H), 3.68-3.73 (m, 4H), 4.10 (q, 4H, J=7.0 Hz), 5.95 (d, 2H, J=13.4 Hz), 7.12-7.18 (m, 2H), 7.23 (d, 2H, J=7.8 Hz), 7.33-7.39 (m, 2H), 7.53 (d, 2H, J=7.3 Hz), 7.61 (d, 2H, J=13.4 Hz). ¹³C NMR (400 MHZ, DMSO-d₆): δ 12.1, 21.8, 25.0, 28.7, 38.3, 46.9, 48.1, 54.7, 96.2, 110.1, 122.7, 123.6, 124.1, 128.8, 140.6, 140.7, 142.6, 168.2. LC MS (m z): 560.9 (M⁺).

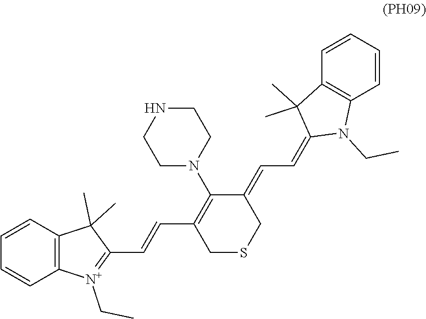 [SMILES: CCN1/C(=C/C=C2\CSCC(/C=C/C3=[N+](CC)c4ccccc4C3(C)C)=C2N2CCNCC2)C(C)(C)c2ccccc21]

### 1-ethyl-2-((E)-2-((Z)-5-(2-((E)-1-ethyl-3,3-dimethylindolin-2-ylidene)-ethylidene)-4-(piperazin-1-yl)-5,6-dihydro-2H-thiopyran-3-yl) vinyl)-3,3-dimethyl-3H-indol-1-ium iodide

PH01 (Z=S) was achieved by the condensation reaction between modified Vilsmeier-Haack reagent and the heterocyclic 3H-indolium salt 1 (R=Et) under basic condition. The final product PH09 was accomplished by the substitution of meso chlorine atom from the heptamethine cyanine dye PH01 (Z=S) by the piperazine in hot DMF. Each step of the reaction was monitored by TLC. But the formation of heptamethine cyanine dye PH01 (Z=S) and piperazine derivative of heptemethine cyanine fluorophore PH09 were monitored by both TLC, and UV-vis spectrometry. The final product PH09 was purified by precipitation using methonal/diethyl ether and column chromatography using 10-15% acetone in DCM as an eluent. Yield (28%, 0.180 g). Mp 175-180° C.; ¹H NMR (400 MHZ, DMSO-d₆): δ ppm 1.25 (br, 6H), 1.6 (S, 12H), 3.08 (br, 4H), 3.54 (br, 4H), 3.89 (br, 4H), 4.01 (br, 4H), 5.85 (br, 2H), 7.11 (br, 4H), 7.11 (s, 2H), 7.43 (br, 4H) ¹³C NMR (100 MHZ, DMSO-d₆): δ ppm 12.03, 23.43, 28.91, 37.71, 47.50, 56.08, 92.82, 109.27, 118.02, 122.71, 139.90, 140.33, 142.90, 167.47; λabs=605 nm in MeOH; HRMS (ESI) Calcd. for [C₃₈H₄₆Cl₃N₂]¹⁺ m/z 579.87, found m/z 579.3516.

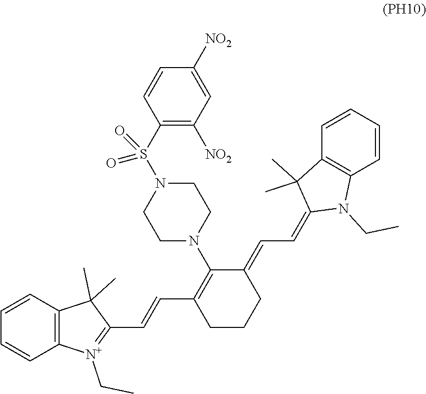 [SMILES: CCN1/C(=C/C=C2\CCCC(/C=C/C3=[N+](CC)c4ccccc4C3(C)C)=C2N2CCN(S(=O)(=O)c3ccc([N+](=O)[O-])cc3[N+](=O)[O-])CC2)C(C)(C)c2ccccc21]

### 2-((E)-2-((E)-2-(4-((2,4-dinitrophenyl) sulfonyl) piperazin-1-yl)-3-(2-((E)-1-ethyl-3,3-dimethylindolin-2-ylidene)ethylidene) cyclohex-1-en-1-yl) vinyl)-1-ethyl-3,3-dimethyl-1-indol-1-ium, iodide

To an anhydrous solution of PH08 (230 mg, 0.40 mmol) in dichloromethane and acetone (1:1), 2,4-dinitrobenzenesulfonyl chloride (1.2 equiv.) was added portion wise at 0° C. in the presence of a catalytic amount of triethylamine under a nitrogen atmosphere. After completion of the reaction as confirmed by TLC and UV, the reaction mixture was cooled to room temperature and the solvents were removed under reduced pressure. The crude product was purified by silica gel column chromatography eluted with CH₂Cl₂/CH₃OH (9:1, v/v). The pure product was obtained in an 80% yield. Mp: 219-222° C. ¹H NMR (400 MHZ, DMSO-d₆): 1.26 (t, 6H, J=6.5 Hz), 1.51 (s, 12H), 1.71-1.79 (m, 2H), 3.49-3.53 (m, 4H), 3.59-3.69 (m, 4H), 4.08-4.19 (m, 4H), 6.05 (d, 2H, J=13.5 Hz), 7.17-7.23 (m, 2H), 7.31-7.41 (m, 4H), 7.50 (d, 2H, J=13.5 Hz), 8.42 (d, 1H, J=8.8 Hz), 7.73-7.83 (dd, 1H, J=2.5 Hz, 8.8 Hz), 9.28 (d, 1H, J=2.5 Hz). ¹³C NMR (400 MHZ, DMSO-d₆): 12.4, 25.1, 28.1, 30.9, 48.6, 50.9, 53.4, 87.8, 110.8, 122.8, 125.8, 141.1, 141.9, 142.2, 148.5, 151.2, 169.7. LC MS (m z): 790 (M⁺−1).

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (I): 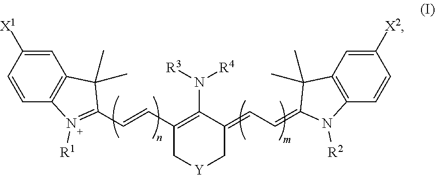
or a pharmaceutically acceptable salt thereof, wherein:
Y is selected from CH₂, O, and S;
X¹ and X² are each independently selected from H, halogen, CN, NO₂, OH, NH₂, C₁₋₃ alkyl, C₁₋₃ haloalkyl, C₁₋₃ alkoxy, C₁₋₃ haloalkoxy, C₁₋₃ alkylamino, and di(C₁₋₃ alkyl)amino;
n is 1 or 2; m is 1 or 2;
R′ and R² are each independently selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl;
R³ and R⁴ are each independently selected from H, C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl;
provided that at least one of R³ and R⁴ is other than H; or
R³ and R⁴ together with the N atom to which they are attached form a ring of formula: 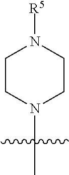
wherein R⁵ is selected from H, C₁₋₈ alkyl, C₁₋₈ haloalkyl, C(═O)Cy¹, and S(═O)₂Cy¹, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, or Cy¹; and
each Cy¹ is independently selected from C₆₋₁₀ aryl and 5-10 membered heteroaryl, each of which is optionally substituted with 1 or 2 substituents independently selected from NO₂, CN, OH, C₁₋₃ alkoxy, C(═O)OH, NH₂, C₁₋₃ alkylamino, and di(C₁₋₃ alkyl)amino.

2. The compound of claim 1, wherein the compound has formula: 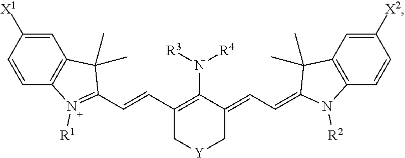
or a pharmaceutically acceptable salt thereof.

3. The compound of claim 1, wherein n is 1 and m is 2, or n is 2 and m is 1.

4. The compound of claim 1, wherein Y is CH₂.

5. The compound of claim 1, wherein Y is O.

6. The compound of claim 1, wherein Y is S.

7. The compound of claim 1, wherein R¹ is C₁₋₈ alkyl, optionally substituted with OH, C(═O)OH, SO₃H, or C₆₋₁₀ aryl.

8. The compound of claim 1, wherein R² is C₁₋₈ alkyl, optionally substituted with OH, C(═O)OH, SO₃H, or C₆₋₁₀ aryl.

9. The compound of claim 1, wherein X¹ is H and X² is H.

10. The compound of claim 1, wherein X¹ is halogen and X² is halogen.

11. The compound of claim 1, wherein R³ or R⁴ is C₁₋₈ alkyl, optionally substituted with OH, C(═O)OH, SO₃H, or C₆₋₁₀ aryl.

12. The compound of claim 1, wherein R³ and R⁴ together with the N atom to which they are attached form a ring of formula:

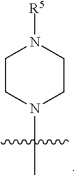

13. The compound of claim 12, wherein R⁵ is selected from H and S(═O)₂Cy¹.

14. The compound of claim 1, selected from any one of the following compounds: 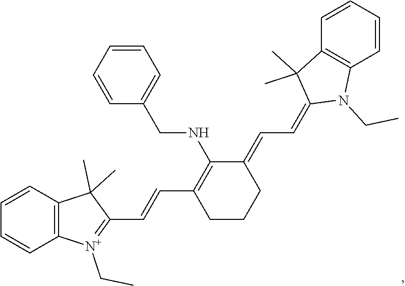 [SMILES: CCN1/C(=C/C=C2\CCCC(/C=C/C3=[N+](CC)c4ccccc4C3(C)C)=C2NCc2ccccc2)C(C)(C)c2ccccc21] 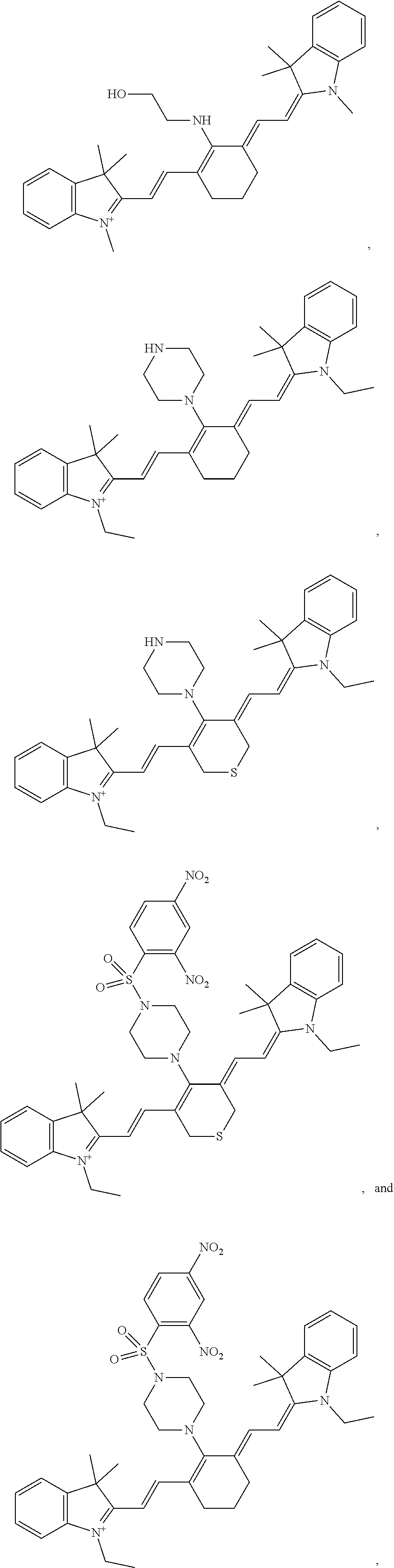 [SMILES: CN1/C(=C/C=C2\CCCC(/C=C/C3=[N+](C)c4ccccc4C3(C)C)=C2NCCO)C(C)(C)c2ccccc21 ; CCN1/C(=C/C=C2\CCCC(/C=C/C3=[N+](CC)c4ccccc4C3(C)C)=C2N2CCNCC2)C(C)(C)c2ccccc21 ; CCN1/C(=C/C=C2\CSCC(/C=C/C3=[N+](CC)c4ccccc4C3(C)C)=C2N2CCNCC2)C(C)(C)c2ccccc21 ; CCN1/C(=C/C=C2\CSCC(/C=C/C3=[N+](CC)c4ccccc4C3(C)C)=C2N2CCN(S(=O)(=O)c3ccc([N+](=O)[O-])cc3[N+](=O)[O-])CC2)C(C)(C)c2ccccc21 ; CCN1/C(=C/C=C2\CCCC(/C=C/C3=[N+](CC)c4ccccc4C3(C)C)=C2N2CCN(S(=O)(=O)c3ccc([N+](=O)[O-])cc3[N+](=O)[O-])CC2)C(C)(C)c2ccccc21]
or a pharmaceutically acceptable salt thereof.

15. A pharmaceutical composition comprising a compound of claim 1, or a pharmaceutically acceptable salt thereof, and a pharmaceutically acceptable carrier.

16. A method of imaging a cancerous tumor in a subject, the method comprising:
i) administering to the subject an effective amount of a compound of claim 1, or a pharmaceutically acceptable salt thereof, or the pharmaceutical composition of claim 15;
ii) waiting a time sufficient to allow the compound to accumulate in the cancerous tumor to be imaged; and
iii) imaging the cancerous tumor with a fluorescence imaging technique.

17. The method of claim 16, wherein said administering comprises topically spraying the cancerous tumor or a site of an organ or tissue comprising the cancerous tumor.

18. The method of claim 16, wherein the fluorescence imaging technique is NIR-II fluorescence imaging.

19. The method of claim 18, wherein the time sufficient to allow the compound to accumulate in the cancerous tumor is from about 1 minute to about 24 hours.

20. A method of treating cancer, the method comprising:
i) imaging a cancerous tumor in a subject according to the method of claim 16; and
ii) surgically removing the cancerous tumor from the subject.
